(19) United States
(12) Patent Application Publication
(10) Pub. No.: US 2014/0196170 A1
(43) Pub. Date: Jul. 10, 2014

(54) ETHYLENE GAS SIGNALING IN PLANTS

(71) Applicant: Salk Institute for Biological Studies, La Jolla, CA (US)

(72) Inventors: Hong Qiao, San Diego, CA (US); Joseph R. Ecker, Carlsbad, CA (US)

(73) Assignee: Salk Institute for Biological Studies, La Jolla, CA (US)

(21) Appl. No.: 14/015,709

(22) Filed: Aug. 30, 2013

Related U.S. Application Data
(60) Provisional application No. 61/695,267, filed on Aug. 30, 2012.

(51) Int. Cl. C12N 15/82

(52) U.S. Cl. CPC C12N 15/8271
USPC 800/278; 800/298; 800/320.2; 800/320.1; 800/320.3; 800/320; 800/317.4; 800/317.2; 800/309; 800/317.3; 800/305; 800/306; 800/314; 800/312; 800/313; 800/307; 800/317; 800/317.1; 800/310; 800/316; 800/315; 800/323.3; 800/323.2; 435/320.1

(57) ABSTRACT

Provided herein are, inter alia, transgenic plants with altered ethylene sensitivity. The transgenic plants provided herein express an EIN2 protein including an amino acid mutation at a position corresponding to position 645 of SEQ ID NO:1. Expression of the EIN2 protein carrying the mutation at position 645 will result in plants with modulated ethylene sensitivity. In some embodiments, the mutation at position 645 of the EIN2 protein will result in plants with increased ethylene sensitivity. Alternatively, in other embodiments, the mutation at position 645 of the EIN2 protein will result in plants with decreased ethylene sensitivity.

### CROSS-REFERENCES TO RELATED APPLICATIONS

The present patent application claims benefit of priority to U.S. Provisional Patent Application No. 61/695,267, filed Aug. 30, 2012, which is incorporated herein by reference and for all purposes.

### STATEMENT AS TO RIGHTS TO INVENTIONS MADE UNDER FEDERALLY SPONSORED RESEARCH AND DEVELOPMENT

This invention was made with government support under F32-HG004830 awarded by the National Institutes of Health. The Government has certain rights in the invention.

### REFERENCE TO A “SEQUENCE LISTING,” A TABLE, OR A COMPUTER PROGRAM LISTING APPENDIX SUBMITTED ON A COMPACT DISK

The Sequence Listing written in file 92150-886240_ST25.TXT, created on Aug. 28, 2013, 200,424 bytes, machine format IBM-PC, MS-Windows operating system, is hereby incorporated by reference in its entirety for all purposes.

### BACKGROUND OF THE INVENTION

The plant hormone ethylene (C₂H₄) is essential for a myriad of physiological and developmental processes. Molecular genetic dissection has revealed that ethylene is perceived by a family of the endoplasmic reticulum (ER)-membrane-bound receptors that are similar in sequence and structure to bacterial two-component histidine kinases (1-4). Each receptor has an amino-terminal transmembrane domain that binds ethylene via a copper cofactor, most likely provided by the copper transporter, RESPONSIVE TO ANTAGONIST1 (5). Signaling from one of the receptors ETR1 (ETHYLENE RESPONSE1) is promoted by interacting with another ER-localized protein REVERSION TO ETHYLENE SENSITIVITY1 (6). The ethylene receptors function redundantly (2) via CTR1 (CONSTITUTIVE TRIPLE RESPONSE-1), a downstream Raf-like protein kinase (7, 8). CTR1 is also associated with the ER membrane, where it directly interacts with ETR1 (8, 9). Downstream of CTR1 is EIN2 (ETHYLENE INSENSITIVE2) (10, 11), an essential positive regulator of ethylene signaling, that shares sequence identity at its amino-terminus with the 12-transmembrane domain of the NRAMP family of metal transporters, and contains a large ˜800 amino acid carboxyl-terminal domain (CEND) (11). Previous studies using heterologous expression of Arabidopsis EIN2 in N. benthamiana suggested that EIN2 might be localized to the ER where it can interact with ETR1 (12). Furthermore, EIN2 is targeted by F-box proteins EIN2INTERACTING PROTEIN1 and EIN2-INTERACTING PROTEIN2, which mediates protein degradation of EIN2 via the ubiquitin-proteasome pathway in the absence of ethylene (13). In an unknown fashion, EIN2 transduces signals to transcription factors EIN3/EIL1 (ETHYLENE INSENSITIVE3/ETHYLENE INSENSITIVE LIKE1), which are sufficient and necessary for activation of all ethylene-response genes (14). A model for hormone signaling has emerged in which perception of ethylene by the receptors alters the activity of CTR1, which in turn by an unknown mechanism, functions to relieve repression of EIN2, resulting in activation of EIN3/EIL1-dependent transcription and activation of ethylene response.

### BRIEF SUMMARY OF THE INVENTION

Provided herein are, inter alia, transgenic plants with altered ethylene sensitivity. The transgenic plants provided herein express an EIN2 protein including an amino acid mutation at a position corresponding to position 645 of SEQ ID NO:1. Expression of the EIN2 protein carrying the mutation at position 645 will result in plants with modulated ethylene sensitivity. In some embodiments, the mutation at position 645 of the EIN2 protein will result in plants with increased ethylene sensitivity. Alternatively, in other embodiments, the mutation at position 645 of the EIN2 protein will result in plants with decreased ethylene sensitivity.

Accordingly, in one aspect a non-naturally occurring plant expressing an EIN2 protein including an amino acid mutation at a position corresponding to position 645 of SEQ ID NO:1 is provided, and the non-naturally occurring plant has modulated ethylene sensitivity compared to a wildtype plant. In embodiments, the EIN2 protein is at least 80% (e.g., 85%, 90%, 95%, 98%) identical to one of SEQ ID NOs:1-18. In embodiments, the EIN2 protein is substantially identical (e.g., at least 80%, 85%, 90%, 95% or 100% identical) to any one of SEQ ID NOs:1-18. In embodiments, the amino acid mutation mimics an unphosphorylated serine. Where the amino acid mutation mimics an unphosphorylated serine, the amino acid mutation may be a serine to alanine mutation. In embodiments, the amino acid mutation is a serine to glycine mutation. In embodiments, the amino acid mutation is a serine to valine mutation. In embodiments, the amino acid mutation is a serine to leucine mutation. In embodiments, expressing the EIN2 protein increases ethylene sensitivity of the non-naturally occurring plant compared to a wildtype plant. In embodiments, the amino acid mutation mimics a phosphorylated serine. Where the amino acid mutation mimics a phosphorylated serine, the amino acid mutation is a serine to glutamic acid mutation. In embodiments, the amino acid mutation is a serine to aspartic acid mutation. In embodiments, the EIN2 protein decreases ethylene sensitivity of the non-naturally occurring plant compared to a wildtype plant.

The transgenic plants provided herein may include a recombinant nucleic acid encoding an EIN2 protein including an amino acid mutation at position 645. In embodiments, the recombinant nucleic acid includes at least 20 (e.g., at least 50, 100, or 200) contiguous nucleotides of a nucleic acid encoding any of the proteins of SEQ ID NOs:1-18. In some embodiments, the nucleic acid includes a sequence at least 80% identical to at least 100 contiguous nucleotides of a nucleic acid encoding any of the proteins of SEQ ID NOs:1-18. In some embodiments, the nucleic acid is at least 95% identical to at least 100 contiguous nucleotides of a nucleic acid encoding any of the proteins of SEQ ID NOs:1-18. In some embodiments, the nucleic acid is 100% identical to at least 100 contiguous nucleotides of a nucleic acid encoding any of the proteins of SEQ ID NOs:1-18. In embodiments, the EIN2 protein is at least 80% (e.g., 85%, 90%, 95%, 98%) identical to one of SEQ ID NOs:1-18.

In embodiments, the recombinant nucleic acid forms part of an expression cassette. Thus, in embodiments, the EIN2 protein is encoded by a nucleic acid operably linked to an inducible promoter. In embodiments, the EIN2 protein is encoded by a nucleic acid operably linked to a tissue-specific promoter. In other embodiments, the EIN2 protein is encoded by a nucleic acid operably linked to an endogenous promoter or an exogenous promoter. In embodiments, the plant may be a transgenic plant.

Alternatively, the non-naturally occurring plant expressing an EIN2 protein including an amino acid mutation at a position corresponding to position 645 of SEQ ID NO:1 may be formed by genome editing. Thus, in embodiments, the non-naturally occurring plant provided herein including embodiments thereof may include an edited genome. In embodiments, the genome is edited at a position corresponding to position 645 of SEQ ID NO:1. In embodiments, the nucleic acid encoding the amino acid mutation is introduced into the plant genome by genome editing. In embodiments, the nucleic acid encoding the amino acid mutation is introduced into the plant genome by CRISPR. Where genome editing technologies (e.g., CRISPR) are used to form a plant expressing an EIN2 protein including an amino acid mutation corresponding to position 645, expression of the EIN2 protein may be controlled by an endogenous promoter. In embodiments, the EIN2 protein is at least 80% (e.g., 85%, 90%, 95%, 98%) identical to one of SEQ ID NOs:1-18. In embodiments, the EIN2 protein is substantially identical (e.g., at least 80%, 85%, 90%, 95% or 100% identical) to any one of SEQ ID NOs:1-18.

In embodiments, the plant is selected from the group consisting of rice, maize, wheat, barley, sorghum, millet, grass, moss, oats, tomato, potato, legume, banana, kiwi fruit, avocado, melon, mango, cane, sugar beet, tobacco, papaya, peach, strawberry, raspberry, blackberry, blueberry, lettuce, cabbage, cauliflower, onion, broccoli, brussels sprouts, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, castor oil plant, spinach, apples, cherries, cranberries, grapefruit, lemons, limes, nectarines, oranges, pears, tangelos, tangerines, lily, carnation, chrysanthemum, petunia, rose, geranium, violet, gladioli, orchid, lilac, crabapple, sweetgum, maple, poinsettia, locust, ash, linden tree, poplar tree and Arabidopsis thaliana. In other embodiments, the plant is selected from the group consisting of Arabidopsis thaliana, melon, legume, rice, petunia, poplar tree, peach and tomato. In some embodiments, the plant is selected from the group consisting of Arabidopsis thaliana, melon, carnation, legume, peach, castor oil plant, tomato, sorghum, corn and selaginella.

In another aspect, a non-naturally occurring plant expressing an EIN2 protein including a serine to alanine mutation at a position corresponding to position 645 of SEQ ID NO:1. In embodiments, the EIN2 protein is at least 80% (e.g., 85%, 90%, 95%, 98%) identical to one of SEQ ID NOs:1-18. In embodiments, the EIN2 protein is substantially identical (e.g., at least 80%, 85%, 90%, 95% or 100% identical) to any one of SEQ ID NOs:1-18. In embodiments, the plant is selected from the group consisting of rice, maize, wheat, barley, sorghum, millet, grass, moss, oats, tomato, potato, legume, banana, kiwi fruit, avocado, melon, mango, cane, sugar beet, tobacco, papaya, peach, strawberry, raspberry, blackberry, blueberry, lettuce, cabbage, cauliflower, onion, broccoli, brussels sprouts, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, castor oil plant, spinach, apples, cherries, cranberries, grapefruit, lemons, limes, nectarines, oranges, pears, tangelos, tangerines, lily, carnation, chrysanthemum, petunia, rose, geranium, violet, gladioli, orchid, lilac, crabapple, sweetgum, maple, poinsettia, locust, ash, linden tree, poplar tree and Arabidopsis thaliana. In embodiments, the plant is selected from the group consisting of Arabidopsis thaliana, melon, legume, rice, petunia, poplar tree, peach and tomato. In embodiments, the plant is selected from the group consisting of Arabidopsis thaliana, melon, carnation, legume, peach, castor oil plant, tomato, sorghum, corn and selaginella.

In one aspect, a non-naturally occurring plant expressing an EIN2 protein including a serine to glutamic acid mutation at a position corresponding to position 645 of SEQ ID NO:1 is provided. In embodiments, the EIN2 protein is at least 80% (e.g., 85%, 90%, 95%, 98%) identical to one of SEQ ID NOs:1-18. In embodiments, the EIN2 protein is substantially identical (e.g., at least 80%, 85%, 90%, 95% or 100% identical) to any one of SEQ ID NOs:1-18. In embodiments, the plant is selected from the group consisting of rice, maize, wheat, barley, sorghum, millet, grass, moss, oats, tomato, potato, legume, banana, kiwi fruit, avocado, melon, mango, cane, sugar beet, tobacco, papaya, peach, strawberry, raspberry, blackberry, blueberry, lettuce, cabbage, cauliflower, onion, broccoli, brussels sprouts, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, castor oil plant, spinach, apples, cherries, cranberries, grapefruit, lemons, limes, nectarines, oranges, pears, tangelos, tangerines, lily, carnation, chrysanthemum, petunia, rose, geranium, violet, gladioli, orchid, lilac, crabapple, sweetgum, maple, poinsettia, locust, ash, linden tree, poplar tree and Arabidopsis thaliana. In other embodiments, the plant is selected from the group consisting of Arabidopsis thaliana, melon, legume, rice, petunia, poplar tree, peach and tomato. In other embodiments, the plant is selected from the group consisting of Arabidopsis thaliana, melon, carnation, legume, peach, castor oil plant, tomato, sorghum, corn and selaginella.

Provided herein are recombinant expression cassettes for the expression of an EIN2 protein including an amino acid mutation at a position corresponding to position 645 of SEQ ID NO:1. Thus, in one aspect, a recombinant expression cassette including a promoter operably linked to a nucleic acid encoding an EIN2 protein is provided and the EIN2 protein includes an amino acid mutation at a position corresponding to position 645 of SEQ ID NO:1. In embodiments, the EIN2 protein is at least 80% (e.g., 85%, 90%, 95%, 98%) identical to one of SEQ ID NOs:1-18. In embodiments, the EIN2 protein is substantially identical (e.g., at least 80%, 85%, 90%, 95% or 100% identical) to any one of SEQ ID NOs:1-18. In embodiments, the nucleic acid includes at least 20 (e.g., at least 50, 100, or 200) contiguous nucleotides of a nucleic acid encoding any of the proteins of SEQ ID NOs:1-18. In some embodiments, the nucleic acid includes a sequence at least 80% identical to at least 100 contiguous nucleotides of a nucleic acid encoding any of the proteins of SEQ ID NOs:1-18. In some embodiments, the nucleic acid is at least 95% identical to at least 100 contiguous nucleotides of a nucleic acid encoding any of the proteins of SEQ ID NOs:1-18. In some embodiments, the nucleic acid is 100% identical to at least 100 contiguous nucleotides of a nucleic acid encoding any of the proteins of SEQ ID NOs:1-18. In embodiments, the EIN2 protein is at least 80% (e.g., 85%, 90%, 95%, 98%) identical to one of SEQ ID NOs:1-18.

In embodiments, the amino acid mutation mimics an unphosphorylated serine. Where the amino acid mutation mimics an unphosphorylated serine, the amino acid mutation may be a serine to alanine mutation. In embodiments, the EIN2 protein increases ethylene sensitivity in a plant expressing the recombinant expression cassette compared to a control plant lacking the expression cassette.

In embodiments, the amino acid mutation mimics a phosphorylated serine. In embodiments, the amino acid mutation is a serine to glutamic acid mutation. In embodiments, the EIN2 protein decreases ethylene sensitivity in a plant expressing the recombinant expression cassette compared to a control plant lacking the expression cassette.

In embodiments, the promoter is an inducible promoter. In embodiments, the promoter is a tissue-specific promoter. In embodiments, the promoter is an endogenous promoter or an exogenous promoter.

In another aspect, a recombinant nucleic acid encoding an EIN2 protein including a serine to alanine mutation at a position corresponding to position 645 of SEQ ID NO:1 is provided. In embodiments, the EIN2 protein is at least 80% (e.g., 85%, 90%, 95%, 98%) identical to one of SEQ ID NOs:1-18. In embodiments, the EIN2 protein is substantially identical (e.g., at least 80%, 85%, 90%, 95% or 100% identical) to any one of SEQ ID NOs:1-18. In embodiments, the nucleic acid includes at least 20 (e.g., at least 50, 100, or 200) contiguous nucleotides of a nucleic acid encoding any of the proteins of SEQ ID NOs:1-18. In some embodiments, the nucleic acid includes a sequence at least 80% identical to at least 100 contiguous nucleotides of a nucleic acid encoding any of the proteins of SEQ ID NOs:1-18. In some embodiments, the nucleic acid is at least 95% identical to at least 100 contiguous nucleotides of a nucleic acid encoding any of the proteins of SEQ ID NOs:1-18. In some embodiments, the nucleic acid is 100% identical to at least 100 contiguous nucleotides of a nucleic acid encoding any of the proteins of SEQ ID NOs:1-18. In embodiments, the EIN2 protein is at least 80% (e.g., 85%, 90%, 95%, 98%) identical to one of SEQ ID NOs:1-18.

In another aspect, a recombinant nucleic acid encoding an EIN2 protein including a serine to glutamic acid mutation at a position corresponding to position 645 of SEQ ID NO:1 is provided. In embodiments, the EIN2 protein is at least 80% (e.g., 85%, 90%, 95%, 98%) identical to one of SEQ ID NOs:1-18. In embodiments, the EIN2 protein is substantially identical (e.g., at least 80%, 85%, 90%, 95% or 100% identical) to any one of SEQ ID NOs:1-18. In embodiments, the nucleic acid includes at least 20 (e.g., at least 50, 100, or 200) contiguous nucleotides of a nucleic acid encoding any of the proteins of SEQ ID NOs:1-18. In some embodiments, the nucleic acid includes a sequence at least 80% identical to at least 100 contiguous nucleotides of a nucleic acid encoding any of the proteins of SEQ ID NOs:1-18. In some embodiments, the nucleic acid is at least 95% identical to at least 100 contiguous nucleotides of a nucleic acid encoding any of the proteins of SEQ ID NOs:1-18. In some embodiments, the nucleic acid is 100% identical to at least 100 contiguous nucleotides of a nucleic acid encoding any of the proteins of SEQ ID NOs:1-18. In embodiments, the EIN2 protein is at least 80% (e.g., 85%, 90%, 95%, 98%) identical to one of SEQ ID NOs:1-18.

In one aspect, a method of making a plant as provided herein including embodiments thereof is provided. The method includes, introducing a nucleic acid encoding an EIN2 protein including an amino acid mutation at a position corresponding to position 645 of SEQ ID NO:1 into a plurality of plants and selecting a plant that expresses the EIN2 protein from the plurality of plants. In embodiments, the selecting step includes selecting a plant that has altered ethylene sensitivity. In embodiments, the EIN2 protein is at least 80% (e.g., 85%, 90%, 95%, 98%) identical to one of SEQ ID NOs:1-18. In embodiments, the EIN2 protein is substantially identical (e.g., at least 80%, 85%, 90%, 95% or 100% identical) to any one of SEQ ID NOs:1-18.

Other inventions provided herein will be clear upon review of the rest of the specification and claims.

### DEFINITIONS

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that may be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

An “EIN2 polypeptide” or “EIN2 protein” is a polypeptide substantially identical to any of SEQ ID NOs:1-18. An EIN2 protein is an essential positive regulator of ethylene signaling, that shares sequence identity at its amino-terminus with the 12-transmembrane domain of the NRAMP family of metal transporters, and contains a large ˜800 amino acid carboxyl-terminal domain (CEND). An “EIN2 protein” as provided herein includes any of the naturally-occurring forms of the EIN2 protein or variants, homologs or functional fragments thereof that maintain EIN2 protein activity (e.g. at least 50%, 80%, 90%, 95%, 96%, 97%, 98%, 99% or 100% activity compared to EIN2). In some aspects, variants have at least 90%, 95%, 96%, 97%, 98%, 99% or 100% amino acid sequence identity across the whole sequence or a portion of the sequence (e.g. a 50, 100, 150 or 200 continuous amino acid portion, e.g., the CEND domain of EIN2) compared to a naturally occurring EIN2 polypeptide. In some aspects, the EIN2 protein is the protein of SEQ ID NOs:1-18.

The “ethylene response” or “ethylene sensitivity” refers to a plant trait that is mediated by ethylene gas, including but not limited to germination, flower and leaf senescence, fruit ripening, fruit drop, leaf abscission, root nodulation, programmed cell death, responsiveness to stress, responsiveness to pathogen attack, and the “triple response” of etiolated dicotyledoneous seedlings (e.g., inhibition of hypocotyl and root cell elongation, radial swelling of the hypocotyl, and exaggerated curvature of the apical hook). Ethylene causes developmental changes that result in fruit ripening. New enzymes are made because of the ethylene signal. These include hydrolases to facilitate break down of fruit components, amylases to accelerate hydrolysis of starch into sugar, pectinases to catalyze degradation of pectin, and so on. Ethylene increases the transcription of genes that are then transcribed and translated to make these enzymes. The enzymes then catalyze reactions to alter the characteristics of the fruit. Enzymes produced as a result of exposure to ethylene facilitate the ripening responses. Chlorophyll is broken down and sometimes new pigments are made so that the fruit skin changes color from green to red, yellow, or blue. Acids are broken down so that the fruit changes from sour to neutral. The degradation of starch by amylase produces sugar. This reduces the mealy (floury) quality and increases juiciness of the fruit. The breakdown of pectin by pectinase results in a softer fruit. Enzymes also break down large organic molecules into volatile smaller molecules which are detected as an aroma.

Fruit drop is related to fruit ripening. The fruit-ripening process described above, also occurs in a layer of cells in the pedicel near the point of attachment to the stem of the plant. This layer of cells in the pedicel is often called the abscission zone because this layer will eventually separate and the fruit will drop from the plant. The cells in this cross sectional layer in the pedicel receive the ethylene signal from the ripening fruit. Reception of the signal results in the production of new enzymes. The cells “ripen” and pectinases attack the cells of the abscission zone. When the cell connection have been sufficiently weakened, the weight of the fruit will cause it to fall from the plant.

Plant senescence is a genetically programmed process; it is the last phase of plant development and ultimately leads to death. Plant hormones such as ethylene and cytokinins play roles in the regulation of senescence.

One of skill in the art will appreciate that one can test for ethylene sensitivity in a plant in many ways. Increased or decreased ethylene sensitivity is determined in a plant including “an amino acid mutation at position 645 of SEQ ID NO:1 compared to a wildtype (i.e. control) plant.” The wildtype plant will be of the same species and will generally be isogenic compared to the plant comprising the amino acid mutation except for the absence of the amino acid mutation.

“An amino acid mutation that mimics an unphosphorylated serine” as referred to herein is an amino acid (natural or non-natural) present at a defined position (e.g., position 645 of SEQ ID NO:1-18) within a polypeptide (e.g., an EIN2 protein), which confers to said polypeptide the same or similar structural and functional properties an unphosphorylated serine residue at the same position would confer to said polypeptide. Non-limiting example of an amino acid mutation mimicking an unphosphorylated serine are alanine, glycine, valine, leucine, isoleucine and lysine. An alanine residue has similar structure and functionality as a serine with the difference that its chemical structure does not allow for the attachment of a phosphate (PO₄³⁻) group. Therefore, an alanine remains unphosphorylated under conditions, which would result in the phosphorylation of a serine (e.g., the presence of CTRL). In embodiments, the amino acid is an amino acid incapable of binding a phosphate (PO₄³⁻) group. Similarly, “an amino acid mutation that mimics a phosphorylated serine” as referred to herein is an amino acid present at a defined position (e.g., position 645 of SEQ ID NO:1-18) within a polypeptide (e.g., an EIN2 protein), which confers to said polypeptide the same or similar structural and functional properties a phosphorylated serine residue at the same position would confer to said polypeptide. Non-limiting examples of amino acids mimicking a phosphorylated serine are glutamic acid and aspartic acid.

Optimal alignment of sequences for comparison may be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polypeptide sequences means that a polypeptide comprises a sequence that has at least 25% sequence identity. Alternatively, percent identity can be any integer from 25% to 100%. Exemplary embodiments include at least: 25%, 30%, 35%, 40%, 45%, 50%, 55%, 60%, 65%, 70%, 75%, 80%, 85%, 90%, 95%, or 99%. compared to a reference sequence using the programs described herein; preferably BLAST using standard parameters, as described below. Accordingly, EIN2 sequences of the invention include nucleic acid sequences encoding a polypeptide that has substantial identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:18. EIN2 sequences of the invention also include polypeptide sequences having substantial identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17 or SEQ ID NO:18. One of skill will recognize that these values can be appropriately adjusted to determine corresponding identity of proteins encoded by two nucleotide sequences by taking into account codon degeneracy, amino acid similarity, reading frame positioning and the like. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH 7 and the temperature is at least about 60° C.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “promoter” or “regulatory element” refers to a region or sequence determinants located upstream or downstream from the start of transcription and which are involved in recognition and binding of RNA polymerase and other proteins to initiate transcription. Promoters need not be of plant origin, for example, promoters derived from plant viruses, such as the CaMV35S promoter, may be used in the present invention.

A polynucleotide sequence is “heterologous to” a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

An “expression cassette” refers to a nucleic acid construct, which when introduced into a host cell, results in transcription and/or translation of a RNA or polypeptide, respectively. Antisense constructs or sense constructs that are not or cannot be translated are expressly included by this definition.

“Genome editing” as provided herein refers to a genetic engineering process during which DNA is inserted, replaced, or removed from a genome using artificially engineered enzymes (e.g., nucleases). The enzymes create specific double-strand breaks (DSBs) at desired locations in the genome, and harness the cell's endogenous mechanisms to repair the induced break by homologous recombination (HR) and nonhomologous end joining (NHEJ). Non-limiting examples of engineered nucleases useful for genome editing include Zinc finger nucleases (ZFNs), Transcription Activator-Like Effector Nucleases (TALENs), and CRISPR (Clustered Regularly Interspaced Short Palindromic Repeats) nucleases.

### BRIEF DESCRIPTION OF THE DRAWINGS

FIG. 1. The NLS in EIN2 is essential for nuclear localization and the response to ethylene. (FIG. 1A) Wild-type EIN2, but not EIN2 NLS mutations, fully rescue ein2-5. Seedlings were grown for 3 days in the dark without ACC or with ACC. (FIG. 1B) Hypocotyl measurements of 3-day-old etiolated seedlings. Each bar is the average length of at least 15 hypocotyls (error bars indicate mean+/−SD). (FIG. 1C) Confocal images of root cells from 3-day-old etiolated transgenic seedlings treated with or without ACC. (FIG. 1D) Time-lapsed confocal images of a series of root cells expressing EIN2-YFP in 3-day-old etiolated seedlings exposed to 10 ppm ethylene gas. Arrows track specific cell nuclei showing accumulation of EIN2-YFP in response to ethylene.

FIG. 2. Ethylene-stimulated nuclear accumulation of the ER-localized EIN2 requires ETR1 and CTRL but not EIN3/EIL1. (FIG. 2A) Sucrose density-gradient centrifugation was performed by fractionation of microsomal membranes containing Mg2+ or in the absence of Mg2+. ACA2 is an ER marker protein; VM23 is a vacuole membrane marker protein; ATPase is a plasma membrane marker protein. (FIG. 2B) In Arabidopsis root cells, ER-localization of EIN2 in the absence of ACC contrasts with nuclear accumulation in the presence of ACC. (FIG. 2C) Immunofluorescence staining of the subcellular location of ACA2 in Arabidopsis root cells from 3-day-old etiolated seedlings grown with or without ACC. (FIG. 2D) Representative images were acquired from root cells using the same exposure times in all panels. EIN2 immunofluorescence using an anti-EIN2 C-terminus polyclonal antibody, ACA2 immunofluorescence and DAPI staining are shown. Arrows indicate nuclei. Scale bar, 5 microns.

FIG. 3. EIN2 is cleaved and a carboxyl-terminal polypeptide fragment is translocated to the nucleus in response to ethylene. (FIG. 3A) Total proteins were subjected to western blotting with anti-EIN2 and anti-tubulin as a loading control. (FIG. 3B) Total cell membrane and nuclear fractions were prepared and subjected to western blotting with the antiEIN2 and anti-ACA2 or the anti-histone H3 antibody as loading controls. (FIG. 3C) Absolute amounts of each endogenous peptide were obtained by calculating the ratios of light to heavy peptide signals. An asterisk indicates the cleavage site. Sequence Legend: SEQ ID NO:1 (aa630-646).

FIG. 4. CTR1-dependent ethylene-regulated phosphorylation of EIN2 5645 regulates proteolysis and nuclear translocation of EIN2-C′. (FIG. 4.A) Absolute amounts of three EIN2-C′ phosphopeptides before and after treatment with 10 ppm ethylene gas. N/D=not detectable. (FIG. 4.B) Relative phosphorylation levels of EIN2 peptides in wild-type or ctr1-1 plants treated for 4 hrs of air or 10 ppm ethylene gas. Spectral counts were computed by averaging three biological replicates. The total spectral counts from all phosphorylated proteins in each sample are indicated as an internal control. (FIG. 4.C) EIN2-C′ S645A results in constitutive ethylene response phenotypes in dark-grown seedlings. (FIG. 4.D) EIN2 S645A results in constitutive ethylene response phenotypes in 7-week-old plants. (FIG. 4.E) EIN2 S645A plants show transcriptional activation of ethylene responses. (FIG. 4.F) EIN2 S645A plants show constitutive nuclear localization without ethylene from leaf cells. Arrows indicate nuclei. (FIG. 4.G) S645A leads to constitutive cleavage of EIN2. Total proteins from 3-day-old etiolated seedlings were subjected to western blotting using an anti-HA antibody and anti-tubulin as a loading control. EIN2^{S645A/E} represents either full-length EIN2^{S645A} or EIN2^{S645E}. (FIG. 4.H) Purified nuclear proteins prepared from EIN2-YFP-HA plants treated with or without ethylene. Total protein from EIN2^{S645A}-YFP-HA over-expressing plants was prepared and subjected to western blotting using an anti-HA antibody and an anti-histone H3 antibody as a loading control. Scale bar, 5 microns.

FIG. S1. The NLS in EIN2 is essential for nuclear localization and ethylene response. (FIG. S1A) Alignment of partial EIN2 protein sequences from different plant species reveals conservation of a putative nuclear localization sequence (NLS). Dots indicate the position of the predicted NLS sequence. Sequence Legend (in order top to bottom): SEQ ID NOs: 1, 2, 4, 5, 17, 18, 8 and 10. (FIG. S1B) A schematic diagram of the construction of EIN2-GUS. EIN2 fusions with GUS (beta-glucuronidase) reporter protein included the full-length EIN2 protein (upper panel), a 76 amino acid (12191294aa) region contains the wild-type (middle panel) or mutated (lower panel) EIN2 NLS sequence. (FIG. S1C-E) The EIN2 NLS sequence is sufficient for GUS protein localization to the nucleus. (FIG. S1C) GUS staining of tobacco epidermal cells expressing EIN2-C76-GUS (left panel of FIG. S1C and FIG. S1D) and EIN2-C76m-GUS (right panel of FIG. S1C and FIG. S1E). The tobacco leaves were infected with the Agrobacterium containing the constructs indicated in the Figure for 3 days before GUS staining (FIG. S1F) Full-length EIN2-YFP functions normally as wild-type EIN2 and its protein level is up-regulated by ethylene. Total membrane proteins from etiolated seedlings indicated in the Figure were subjected to western blotting and detection using either an anti-GFP or an anti-EIN2 antibody. (FIG. S1G-H) A mutated NLS impaired the nuclear translocation of EIN2. The images were acquired from the root cells of EIN2-YFP (FIG. S1G) or EIN2FmYFP transgenic plants (FIG. S1H) treated with (lower panel) or without (upper panel) ethylene gas. (FIG. S1I) Confocal images of EIN2-YFP expression in root cells upon the exposure to ethylene. Seedlings (3-days-old) were grown in the dark in the presence of hydrocarbon-free air and then were exposed to ethylene gas for different amounts of time. The images were acquired every 30 minutes for 120 minutes. An arrow indicates the localization of nucleus. Scale bar, 5 microns.

FIG. S2. EIN2 is localized to the ER membrane. (FIG. S2A) Subcellular localization of EIN2 in Arabidopsis root cells. (Upper panel) Anti-EIN2 antibody immunofluorescence (IF) staining (white) compared with (lower panel) GFP fluorescence of a known ER-localized marker protein (GFPer, GFP protein with a carboxy-terminal fused ER retention signal—SEKDEL, (24)) in root cells of 4-day-old light grown Arabidopsis (Col-0) seedlings. The arrows indicate localization of EIN2 and GFPer at the cell plate (left-side panels) and the perinuclear-ER (right-side of panels). (FIG. S2B) EIN2-YFP co-localized with the ER marker mCFPer in tobacco epidermal cells. The colors are false pseudo colors. Scale bar, 5 microns. (FIG. S2C) Immunofluorescence staining of EIN2 in the ein2-5 mutant (upper panel) and Col-0 (lower panel) demonstrates the specificity of the EIN2 antibody. Scale bar, 5 microns.

FIG. S3. Nuclear accumulation of the EIN2 carboxyl-terminus is necessary and sufficient to evoke plant ethylene response phenotypes. (FIG. S3A) The phenotype of 8-week-old EIN2-C-YFP transgenic lines is shown (left panel). (FIG. S3B) Confocal image showing subcellular localization of EIN2-C-YFP fluorescence in the nucleus of Arabidopsis root cells of 8-week-old transgenic plants (right panel). “Bright field” indicates an image of the same cells using bright-field microscopy. Arrows indicate the location of nuclei. (FIG. S3C) Confocal images of Arabidopsis cells showing the subcellular location of EIN2-C-YFP-GR fusion protein in root cells of 7-week-old EIN2-C-YFP-GR transgenic plants treated with (+) or without (−) DEX. Arrows indicate the locations of nuclei. (FIG. S3D) Nuclear-localized EIN2-C-YFP induces ethylene response phenotypes. Plants were grown in soil for 7 weeks treated with (right panel) or without (left panel) dexamethasone (DEX). (FIG. S3E) mRNA expression analysis of ERF1 and PDF1.2 in EIN2-C-YFP-GR transgenic plants treated with or without DEX. Total RNA was extracted from the leaves of 7-week-old light-grown plants. The qRT-PCR data were normalized to the corresponding actin (input) controls for all three biological replicates. Double asterisk indicate a significant difference (t-test P<0.001). Scale bar, 5 microns.

FIG. S4. EIN2 is cleaved and a fragment is translocated to the nucleus in response to ethylene. (FIG. S4A) EIN2-C′ accumulates in response to ethylene. 3-day-old etiolated seedlings were grown in the dark with different treatments indicated in the Figure before harvesting tissue. Total protein extractions were subjected to western blotting with an anti-EIN2 antibody to assay the protein level of EIN2-C′-YFP. (FIG. S4B) EIN2-C′YFP accumulated in response to ethylene. Total proteins were isolated from EIN2 YFP/Col-0 transgenic plants treated with or without ethylene gas and were subjected to western blotting with an anti-HA antibody. (FIG. S4C) EIN2-C1 (from 638aa to 1294aa) causes a severe constitutive ethylene response phenotype. 7-week-old plants in soil were photographed. (FIG. S4D) Nuclear localization of EIN2-C1. The images were acquired from the root cells of EIN2-C1-YFP transgenic plants. (FIG. S4E) Pseudo-MRM data of the EIN2 peptides 630-644 (left), 630-645 (middle), and 630-646 (right) after ethylene treatment (SEQ ID NO:1). The top panel plots are the spike-in heavy peptide signals: (left) 714.3->899.5 (b9+), (middle) 757.8->899.5 (b9+), and (right) 831.4->899.5 (b9+); the bottom panel plots are the light (endogenous) peptide signals: (left) 709.3->889.5 (b9+), (middle) 752.8->889.5 (b9+), and (right) 826.4->889.5 (b9+). Scale bar, 5 microns.

FIG. S5. CTR1-dependent ethylene-regulated phosphorylation of EIN2 5645 regulates proteolysis and nuclear translocation of EIN2-C′. (FIG. S5A) Pseudo-MRM data of the EIN2 phosphopeptide (630-AAPTSNFTVGSDGPP[s]FR-647, SEQ ID NO:1 [aa630-647]) before (left) and after (right) ethylene treatment. The top panel plots are the spike-in heavy peptide signal: 949.4->1009.4; the bottom panel plots are the light (endogenous) peptide signal: 944.4->999.4. Notice the y-ions in the heavy labeled peptide (upper spectrum) are all shifted +10 Da due to the C-terminal heavy Arg residue. (FIG. S5B). A protein alignment of part of the EIN2 C-terminal end uncovers the conservation of the phosphorylation sites at S645 and S659/661. Sequence Legend (order of appearance top to bottom: SEQ ID NOs:1, 2, 3, 4, 5, 6, 7, 8, 9, 10, 11, 12, 13, 14, 15, 16. (FIG. S5C-D) The phenotypes of flowers, siliques (FIG. S5C) and seedlings (FIG. S5D) from EIN2^{S645A}-YFP (SEQ ID NO:1) transgenic plants. (FIG. S5E) Identification of EIN2 or EIN2-C′ in transgenic plants expressing EIN2^{S645A} or EIN2^{S645E} mutant proteins. Total proteins from 3-day-old etiolated seedlings of the indicated transgenic lines were subjected to western blotting using an anti-HA antibody for the presence of EIN2 and EIN2-C′, and anti-tubulin antibody as a loading control. EIN2^{S645A/E} represents either full-length EIN2^{S645A} or EIN2^{S645E}.

FIG. S6. Model for the phosphorylation-dependent proteolysis and ER to nucleus translocation of EIN2 C′ polypeptide in response to ethylene. (Left panel) In the absence of hormone, EIN2 is localized in the ER and shows CTRL-dependent phosphorylation, resulting in suppression of ethylene responses. (Right panel) Upon the perception of ethylene gas in the ER by the ethylene receptor ETR1 (25), dephosphorylation of EIN2 at 5645 (SEQ ID NO:1) leads to proteolytic cleavage at this site and release of a large carboxyl-terminal fragment (EIN2-C′), which rapidly translocates to the nucleus and activates EIN3/EIL1-dependent transcription through direct or indirect interaction with EIN3/EIL1. detectable.

### DETAILED DESCRIPTION OF THE INVENTION

### 1. Introduction

Provided herein are, inter alia, methods and compositions for modulating ethylene response in plants. Further provided are non-naturally occurring plants with modulated ethylene sensitivity compared to a wildtype plant. The present invention is based, in part, on the discovery that a serine residue within the CEND of EIN2 (i.e. S645) plays a role in regulating ethylene responses in plants. As described in the Examples, the phosphorylation status of S645 in EIN2 determines EIN2 subcellular location and ethylene sensitivity. More specifically, specific amino acid mutations at position 645 of EIN2 have been shown herein to increase or decrease ethylene sensitivity. These discoveries can now be used to generate plants with increased or decreased ethylene sensitivity as desired.

Those of skill in the art are aware of numerous desirable characteristics associated with decreased ethylene sensitivity. For example, decreased ethylene sensitivity is useful to (a) protect flowers and plants from senescence or deterioration, including but not limited to, when shipped in closed containers, (b) increase the yields of plants by preventing flower abortion, fruit drop and abscission of desirable vegetative parts, and (c) improve the quality of turf by maintaining chlorophyll levels, increasing clipping yields, preventing leaf senescence and increasing disease resistance. Furthermore, a decrease in ethylene response can be used to delay disease developments, including but not limited to preventing of lesions and senescence and to reduce diseases in plants in which ethylene causes an increase in disease development, including but not limited to, in barley, citrus, Douglas fir seedlings, grapefruit, plum, rose, carnation, strawberry, tobacco, tomato, wheat, watermelon and ornamental plants. In some embodiments, decreased ethylene sensitivity is useful for inducing enhanced drought tolerance. Thus, for example, senescence or deterioration may be prevented upon inducement of expression EIN2 including an amino acid mutation at position 645, which mimics phosphorylated serine.

Those of skill in the art are also aware of numerous desirable characteristics associated with increased ethylene sensitivity. Notably, increased ethylene sensitivity can include increased fruit ripening. Thus, for example, ripening can be induced upon inducement of expression EIN2 including an amino acid mutation at position 645, which mimics unphosphorylated serine.

### 2. Use of Nucleic Acids of the Invention to Express EIN2 5645 Mutant Proteins

Nucleic acid sequences encoding all or an active part of an EIN2 polypeptide including an amino acid mutation at position 645 (including but not limited to polypeptides substantially identical to any of SEQ ID NOs:1-18) can be used to prepare expression cassettes that modulate ethylene sensitivity upon expression in a plant.

Any of a number of means well known in the art can be used to express an EIN2 protein including an amino acid mutation at position 645 in plants. Any organ can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit. Alternatively, an EIN2 protein including an amino acid mutation at position 645 can be expressed constitutively (e.g., using the CaMV 35S promoter).

One of skill will recognize that the polypeptides encoded by the genes of the invention, like other proteins, have different domains, which perform different functions. Thus, the gene sequences need not be full length, so long as the desired functional domain of the protein is expressed.

Preparation of Recombinant Vectors

In some embodiments, to use isolated sequences in the above techniques, recombinant DNA vectors suitable for transformation of plant cells are prepared. Techniques for transforming a wide variety of higher plant species are well known and described in the technical and scientific literature. See, for example, Weising et al. Ann. Rev. Genet. 22:421-477 (1988). A DNA sequence coding for the desired polypeptide, for example, a cDNA sequence encoding a full-length protein, will preferably be combined with transcriptional and translational initiation regulatory sequences, which will direct the transcription of the sequence from the gene in the intended tissues of the transformed plant. A variety of different expression constructs, such as expression cassettes and vectors suitable for transformation of plant cells can be prepared. An EIN2 sequence coding for an EIN2 polypeptide including an amino acid mutation at position 645, e.g., a cDNA sequence encoding a full length protein, can be combined with cis-acting (promoter) and trans-acting (enhancer) transcriptional regulatory sequences to direct the timing, tissue type and levels of transcription in the intended tissues of the transformed plant. Translational control elements can also be used.

The invention provides a nucleic acid encoding an EIN2 protein including an amino acid mutation at a position corresponding to position 645 of SEQ ID NO:1, which may be operably linked to a promoter that, in some embodiments, is capable of driving the transcription of the EIN2 coding sequence in plants. The promoter can be, e.g., derived from plant or viral sources. The promoter can be, e.g., constitutively active, inducible, or tissue specific. In construction of recombinant expression cassettes, vectors, transgenics, of the invention, a different promoter can be chosen and employed to differentially direct gene expression, e.g., in some or all tissues of a plant or animal.

For example, for overexpression, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various plant genes known to those of skill.

Alternatively, the plant promoter may direct expression of the polynucleotide of the invention in a specific tissue (tissue-specific promoters) or may be otherwise under more precise environmental control (inducible promoters). Examples of tissue-specific promoters under developmental control include promoters that initiate transcription only in certain tissues, such as fruit, seeds, or flowers. Examples of environmental conditions that may affect transcription by inducible promoters include anaerobic conditions, elevated temperature, or the presence of light.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention can optionally comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

Constitutive Promoters

A promoter fragment can be employed that will direct expression of a nucleic acid encoding an EIN2 protein including an amino acid mutation at position 645 in all transformed cells or tissues, e.g. as those of a regenerated plant. The term “constitutive regulatory element” means a regulatory element that confers a level of expression upon an operatively linked nucleic molecule that is relatively independent of the cell or tissue type in which the constitutive regulatory element is expressed. A constitutive regulatory element that is expressed in a plant generally is widely expressed in a large number of cell and tissue types. Promoters that drive expression continuously under physiological conditions are referred to as “constitutive” promoters and are active under most environmental conditions and states of development or cell differentiation.

A variety of constitutive regulatory elements useful for ectopic expression in a transgenic plant are well known in the art. The cauliflower mosaic virus 35S (CaMV 35S) promoter, for example, is a well-characterized constitutive regulatory element that produces a high level of expression in all plant tissues (Odell et al., Nature 313:810-812 (1985)). The CaMV 35S promoter can be particularly useful due to its activity in numerous diverse plant species (Benfey and Chua, Science 250:959-966 (1990); Futterer et al., Physiol. Plant 79:154 (1990); Odell et al., supra, 1985). A tandem 35S promoter, in which the intrinsic promoter element has been duplicated, confers higher expression levels in comparison to the unmodified 35S promoter (Kay et al., Science 236:1299 (1987)). Other useful constitutive regulatory elements include, for example, the cauliflower mosaic virus 19S promoter; the Figwort mosaic virus promoter; and the nopaline synthase (nos) gene promoter (Singer et al., Plant Mol. Biol. 14:433 (1990); An, Plant Physiol. 81:86 (1986)).

Additional constitutive regulatory elements including those for efficient expression in monocots also are known in the art, for example, the pEmu promoter and promoters based on the rice Actin-1 5′ region (Last et al., Theor. Appl. Genet. 81:581 (1991); Mcelroy et al., Mol. Gen. Genet. 231:150 (1991); Mcelroy et al., Plant Cell 2:163 (1990)). Chimeric regulatory elements, which combine elements from different genes, also can be useful for ectopically expressing a nucleic acid molecule encoding an EIN2 protein including an amino acid mutation at position 645 (Comai et al., Plant Mol. Biol. 15:373 (1990)).

Other examples of constitutive promoters include the 1′- or 2′-promoter derived from T-DNA of Agrobacterium tumefaciens (see, e.g., Mengiste (1997) supra; O'Grady (1995) Plant Mol. Biol. 29:99-108); actin promoters, such as the Arabidopsis actin gene promoter (see, e.g., Huang (1997) Plant Mol. Biol. 1997 33:125-139); alcohol dehydrogenase (Adh) gene promoters (see, e.g., Millar (1996) Plant Mol. Biol. 31:897-904); ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), Gpc2 from maize (GenBank No. U45855, Manjunath et al., Plant Mol. Biol. 33:97-112 (1997)), other transcription initiation regions from various plant genes known to those of skill. See also Holtorf Plant Mol. Biol. 29:637-646 (1995).

Inducible Promoters

Alternatively, a promoter may direct expression of an nucleic acid encoding an EIN2 protein including an amino acid mutation at position 645 of the invention under the influence of changing environmental conditions or developmental conditions. Examples of environmental conditions that may affect transcription by inducible promoters include anaerobic conditions, elevated temperature, drought, or the presence of light. Such promoters are referred to herein as “inducible” promoters. For example, the invention incorporates the drought-inducible promoter of maize (Busk (1997) supra); the cold, drought, and high salt inducible promoter from potato (Kirch (1997) Plant Mol. Biol. 33:897-909).

Alternatively, plant promoters which are inducible upon exposure to plant hormones, such as auxins, are used to express the nucleic acids of the invention. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274:1900-1902).

Promoters that are inducible upon exposure to chemicals reagents applied to the plant, such as herbicides or antibiotics, can also be used to express the nucleic acids of the invention. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. An EIN2 coding sequence can also be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324; Uknes et al., Plant Cell 5:159-169 (1993); Bi et al., Plant J. 8:235-245 (1995)).

Other inducible regulatory elements include but are not limited to copper-inducible regulatory elements (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Roder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)). An inducible regulatory element useful in the transgenic plants of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

Tissue-Specific Promoters

Alternatively, the plant promoter may direct expression of the polynucleotide of the invention in a specific tissue (tissue-specific promoters). Tissue specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, e.g., roots or leaves, or reproductive tissues, such as fruit, ovules, seeds, pollen, pistols, flowers, or any embryonic tissue. Reproductive tissue-specific promoters may be, e.g., ovule-specific, embryo-specific, endosperm-specific, integument-specific, seed and seed coat-specific, pollen-specific, petal-specific, sepal-specific, or some combination thereof.

Other tissue-specific promoters include seed promoters. Suitable seed-specific promoters are derived from the following genes: MAC1 from maize (Sheridan (1996) Genetics 142:1009-1020); Cat3 from maize (GenBank No. L05934, Abler (1993) Plant Mol. Biol. 22:10131-1038); vivparous-1 from Arabidopsis (Genbank No. U93215); atmycl from Arabidopsis (Urao (1996) Plant Mol. Biol. 32:571-57; Conceicao (1994) Plant 5:493-505); napA from Brassica napus (GenBank No. J02798, Josefsson (1987) JBL 26:12196-1301); and the napin gene family from Brassica napus (Sjodahl (1995) Planta 197:264-271).

A variety of promoters specifically active in vegetative tissues, such as leaves, stems, roots and tubers, can also be used to express the nucleic acids encoding an EIN2 protein including an amino acid mutation at position 645 of the invention. For example, promoters controlling patatin, the major storage protein of the potato tuber, can be used, see, e.g., Kim (1994) Plant Mol. Biol. 26:603-615; Martin (1997) Plant J. 11:53-62. The ORF13 promoter from Agrobacterium rhizogenes which exhibits high activity in roots can also be used (Hansen (1997) Mol. Gen. Genet. 254:337-343. Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28:137-144); the curculin promoter active during taro corm development (de Castro (1992) Plant Cell 4:1549-1559) and the promoter for the tobacco root-specific gene TobRB7, whose expression is localized to root meristem and immature central cylinder regions (Yamamoto (1991) Plant Cell 3:371-382).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier (1997) FEBS Lett. 415:91-95). A ribulose bisphosphate carboxylase promoters expressed almost exclusively in mesophyll cells in leaf blades and leaf sheaths at high levels, described by Matsuoka (1994) Plant J. 6:311-319, can be used. Another leaf-specific promoter is the light harvesting chlorophyll a/b binding protein gene promoter, see, e.g., Shiina (1997) Plant Physiol. 115:477-483; Casal (1998) Plant Physiol. 116:1533-1538. The Arabidopsis thaliana myb-related gene promoter (Atmyb5) described by Li (1996) FEES Lett. 379:117-121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize by Busk (1997) Plant J. 11:1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems and are described by Di Laurenzio (1996) Cell 86:423-433; and, Long (1996) Nature 379:66-69, can be used. Another promoter is the 3-hydroxy-3-methylglutaryl coenzyme A reductase HMG2 gene promoter, whose expression is restricted to meristematic and floral (secretory zone of the stigma, mature pollen grains, gynoecium vascular tissue, and fertilized ovules) tissues (see, e.g., Enjuto (1995) Plant Cell. 7:517-527). Additional promoter examples include the knl-related gene promoters from maize and other species that show meristem-specific expression, see, e.g., Granger (1996) Plant Mol. Biol. 31:373-378; Kerstetter (1994) Plant Cell 6:1877-1887; Hake (1995) Philos. Trans. R. Soc. Lond. B. Biol. Sci. 350:45-51. One such example is the Arabidopsis thaliana KNAT1 promoter. In the shoot apex, KNAT1 transcript is localized primarily to the shoot apical meristem; the expression of KNAT1 in the shoot meristem decreases during the floral transition and is restricted to the cortex of the inflorescence stem (see, e.g., Lincoln (1994) Plant Cell 6:1859-1876).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

### 3. Production of Transgenic Plants

In embodiments, the nucleic acid sequences encoding an EIN2 protein including an amino acid mutation at position 645 are expressed recombinantly in plant cells to modulate ethylene sensitivity in the plant. The recombinant nucleic acid encoding an EIN2 protein including an amino acid mutation at position 645 may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Alternatively, methods of genome editing may be applied to introduce an amino acid mutation directly into the genome of a wildtype plant, thereby replacing the corresponding wildtype residue. An example of a genome editing technology well known in the art and contemplated for the invention provided herein is CRISPR (Clustered Regularly Interspaced Short Palindromic Repeats). CRISPR is an RNA-guided genome editing tool frequently used to alter a genomic sequence in vivo. See, for example, Deltcheva et al. Nature 471(7340):602-7 (2011); M. M. Jinek, et al. Science 337, 816-821 (2012); L. A. Marraffini, E. J. Sontheimer, Nature 463, 568 (2010); Wang et al. Cell 153 (4):910-8. (2013). For instance, by using the CRISPR genome editing tool, stretches of genomic coding sequences may be replaced with sequences, which encode one or more amino acid mutations, but are otherwise identical to the sequences being replaced. Thereby, a non-naturally occurring plant endogenously expressing a wildtype EIN2 protein, may upon recombinantly expressing the CRISPR components be transformed into a plant expressing a mutant EIN2 protein.

Microinjection techniques are known in the art and well described in the scientific and patent literature. The introduction of DNA constructs using polyethylene glycol precipitation is described in Paszkowski et al. EMBO J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Agrobacterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983).

Transformed plant cells that are derived from any transformation technique can be cultured to regenerate a whole plant that possesses the transformed genotype and thus the desired phenotype. Such regeneration techniques rely on manipulation of certain phytohormones in a tissue culture growth medium, optionally relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

The nucleic acids of the invention can be used to confer desired traits on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea. Plants having an ethylene response, and thus those that have use in the present invention, include but are not limited to: dicotyledons and monocotyledons including but not limited to rice, maize, wheat, barley, sorghum, millet, grass, oats, tomato, potato, banana, kiwi fruit, avocado, melon, mango, cane, sugar beet, tobacco, papaya, peach, strawberry, raspberry, blackberry, blueberry, lettuce, cabbage, cauliflower, onion, broccoli, brussel sprout, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, spinach, apples, cherries, plums, cranberries, grapefruit, lemons, limes, nectarines, oranges, peaches, pears, tangelos, tangerines, lily, carnation, chrysanthemum, petunia, rose, geranium, violet, gladioli, orchid, lilac, crabapple, sweetgum tree, maple tree, poinsettia, locust tree, oak tree, ash tree and linden tree.

### 4. Examples

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

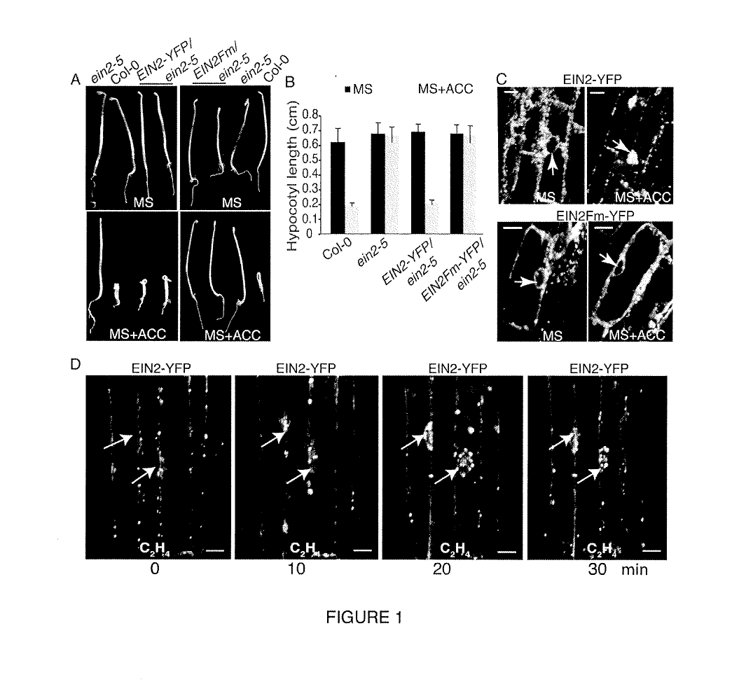

To explore the mechanism of EIN2 function, Applicants identified and tested the requirement of a putative NLS (15) in the evolutionarily conserved EIN2 carboxyl-terminus (FIG. S1A to S1E) and found that a wild-type EIN2-YFP fusion protein maintained its normal function(s) as its expression was able to rescue the ein2-5 mutant phenotype (FIGS. 1A and 1B and FIG. S1F); whereas an NLS mutated EIN2Fm-YFP protein was unable to complement the ein2-5 mutant phenotype (FIG. 1A and FIG. 1B). In the absence of the ethylene precursor ACC (1-aminocyclopropane-1-carboxylate), the EIN2-YFP protein was localized in the ER (FIG. 1C) (12) and accumulated in the nucleus upon exposure to ethylene (FIG. 1C and FIG. S1G). However, nuclear localization of the EIN2Fm-YFP protein was not observed in the presence of ACC (FIG. 1C and FIG. S1H). Therefore, Applicants conclude that the NLS is necessary for EIN2 to function in the ethylene response.

Plants respond to ethylene rapidly and this process is dependent on the function of EIN2 (16). Time-lapsed imaging was used to monitor the dynamics of EIN2 protein movement upon ethylene treatment. In the absence of ethylene (T0), EIN2 protein was absent from the nucleus (FIG. 1D). Nuclear accumulation of EIN2 protein was observed within 10 minutes of exposure to ethylene and protein levels further increased in the nucleus of the same cells during the subsequent 30 minutes (FIG. 1D). A greater amount of EIN2 protein in the nucleus was observed with longer ethylene treatments (FIG. S1I), demonstrating that the ER-nucleus translocation of EIN2 is important in response to ethylene.

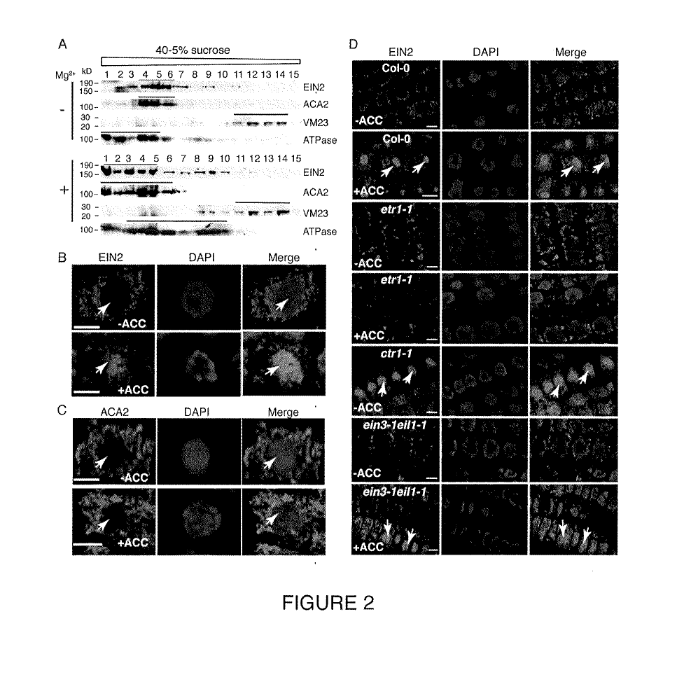

EIN2 protein specifically localized to the ER in the absence of ethylene (FIGS. 2A and 2B, FIG. S2A to S2C), and amassed in the nucleus upon ethylene treatment (FIG. 2B). Whereas a known ER-localized protein was unaffected by ethylene (FIG. 2C). Additionally, in Col-0, EIN2 protein was localized to the ER membrane in the absence of ACC, whereas upon growth on ACC, nuclear translocation of EIN2 protein was observed (FIG. 2D). However, in etr1-1, nuclear accumulation of EIN2 protein was abolished. In contrast, in ctr1-1 a constitutive nuclear accumulation of EIN2 protein was observed, even in the absence of ACC (FIG. 2D). An ein3-1/eil1-1 double mutant had no effect on the nuclear translocation of EIN2 protein (FIG. 2D).

Therefore, Applicants conclude that ETR1 and CTRL are important in the ER-nucleus translocation of EIN2, whereas (EIN3/EIL1) are not required for this process.

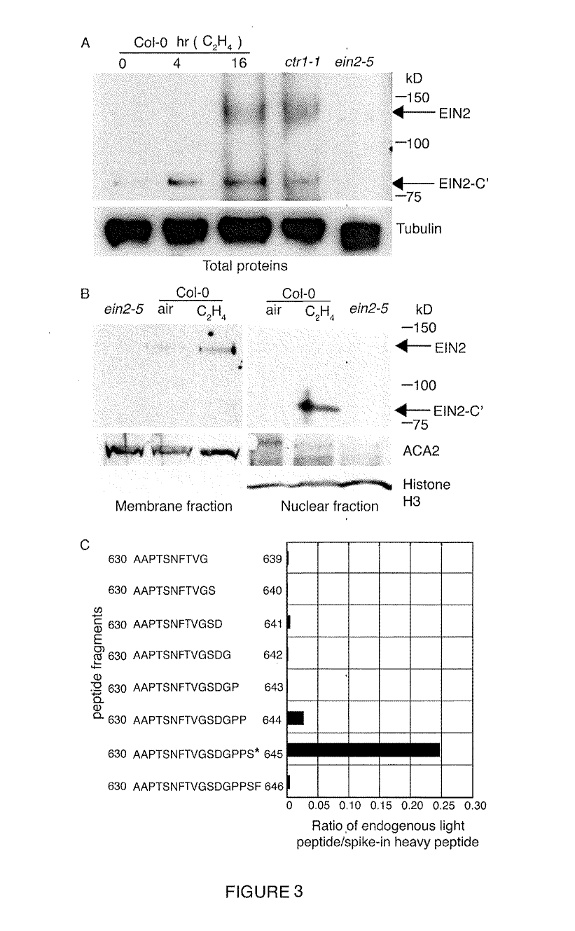

EIN2 is a bifunctional protein (11) and positioning the EIN2-CEND polypeptide in the nucleus was sufficient to mimic both ethylene responses (FIG. S3A to S3E). Applicants hypothesized that EIN2 may be proteolytically processed and its C-terminal fragment translocated to the nucleus upon exposure to ethylene. To test this hypothesis, Applicants first examined the presence of a cleaved form of the native EIN2 protein by western blotting. Although full-length EIN2 was not detectable at 0 and 4 hours of ethylene treatment, it was detected in situations that mimic a constitutive ethylene exposure, such as in the ctr1-1 mutant and in Col-0 plants treated with 16 hours of ethylene. Additionally, Applicants observed the presence of an ˜75 kD carboxyl-terminal EIN2 fragment (called EIN2-C′) whose abundance correlated with the duration of ethylene treatment (FIG. 3A, FIG. S4A and S4B). Although full-length EIN2 was easily detected in the membrane protein fraction (FIG. 3B and (13)), EIN2-C′ was barely detected in the nuclear fraction without ethylene treatment (FIG. 3B). In contrast, the EIN2-C′ polypeptide was readily detected in the nuclear protein fraction treated with ethylene (FIG. 3B). Applicants generated transgenic lines expressing EIN2-C1-YFP, which contained a YFP protein fused to a fragment of EIN2 estimated from the observed EIN2-C′ polypeptide size (638-1294aa), and found EIN2C1-YFP protein was localized to the nucleus exclusively and its expression was sufficient to cause a severe constitutive ethylene response phenotype, reminiscent of ctr1-1 mutants (FIG. S4C and S4D).

Applicants next used mass spectrometry (17) to map the cleavage site of the EIN2 protein. Three EIN2-C′ peptides (630-647aa, 648-662aa, 754-766aa, table S1A) (SEQ ID NO:1) were detected at similar levels in samples treated with air. In the presence of ethylene, the abundance of the peptide (630-647aa) was ˜20-fold less, suggesting that cleavage of EIN2 protein may occur within this peptide. Pseudo-Multiple Reaction Monitoring (18) was used to identify the EIN2 protein cleavage site. Contiguous peptides designated 630-647aa and 648-662aa behaved differently following ethylene treatment; only the former decreased in abundance suggesting that it contained an ethylene-dependent protease cleavage site (table. S1A). Applicants tested a set of overlapping peptides that differed by the progressive loss of single amino acids from the C-terminus of the peptide 630-647aa. These represent 18 possible cleavage products derived from cleaving within residues 630-647 (plus the trypsin cleavage site corresponding to the N-terminus of the peptide). Following ethylene treatment, none of the ten deletions from the N-terminus corresponded to a detectable peptide (table S1B). However, among the eight deletions from the C-terminus, one peptide was significantly enriched (AAPTSNFTVGSDGPPS; 630645aa (SEQ ID NO:1); FIG. 3C, FIG. S4E and table S1B), indicating that ethylene-induced cleavage occurs between 645aa and 646aa.

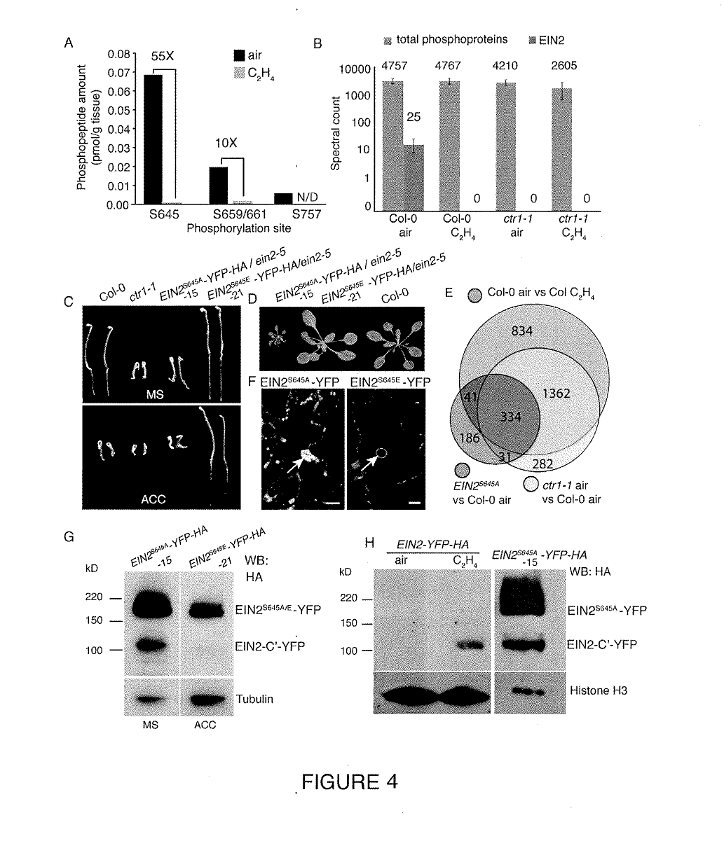

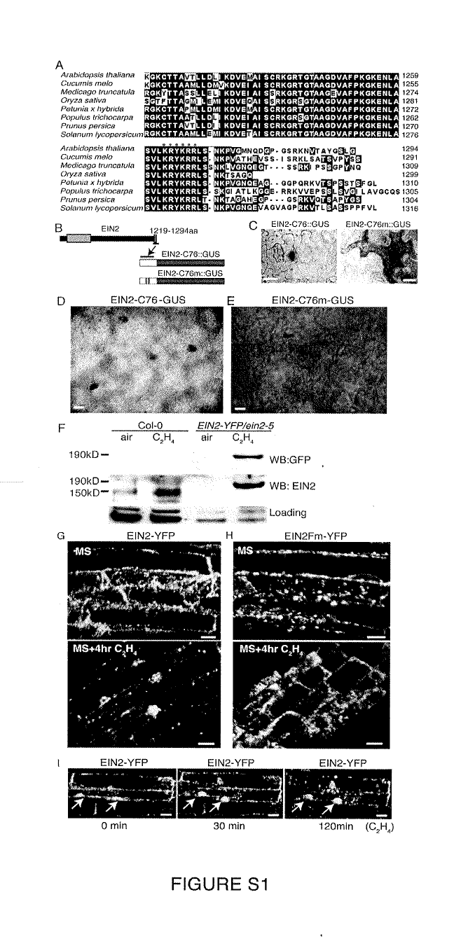

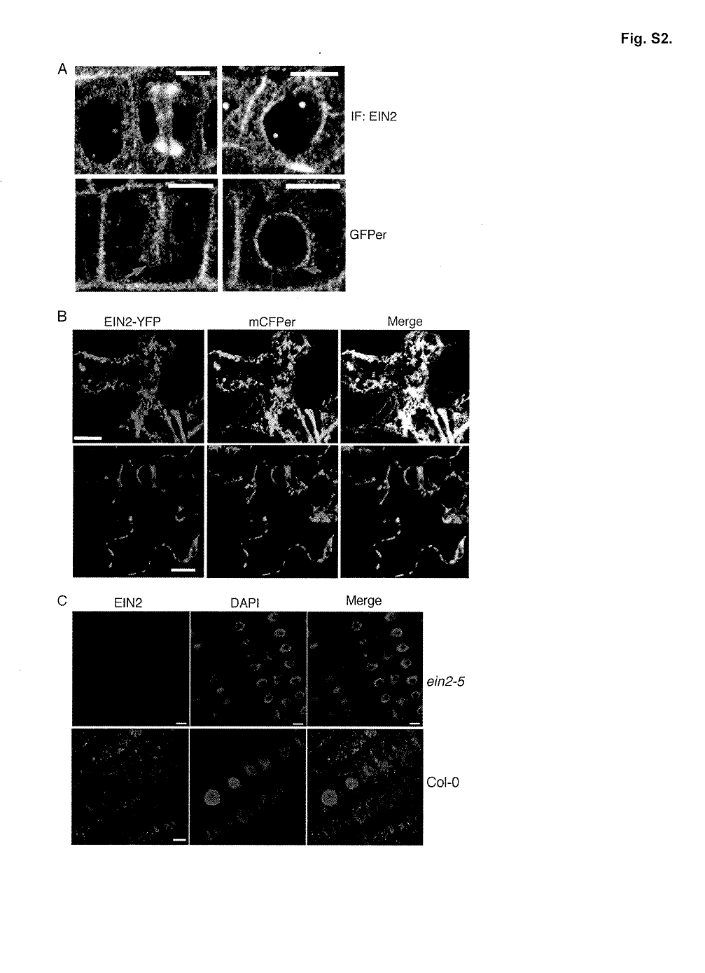

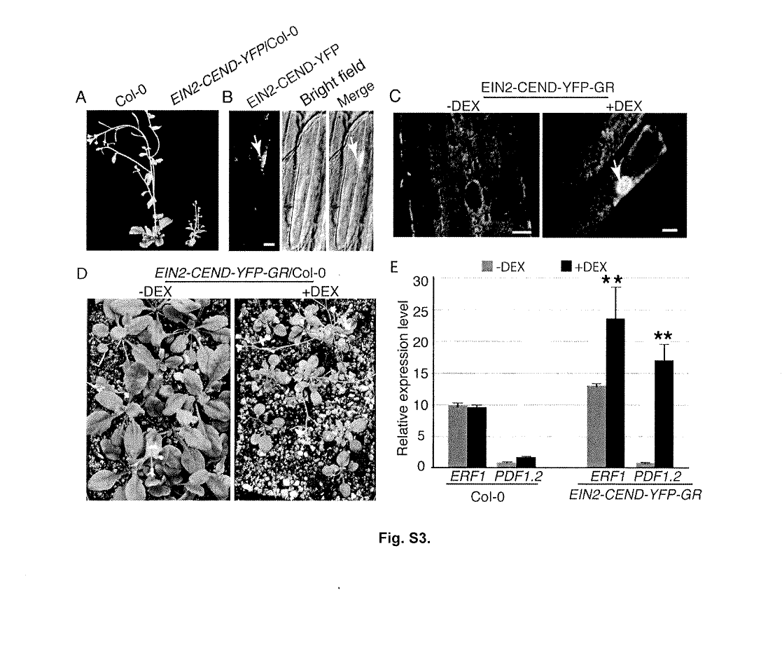

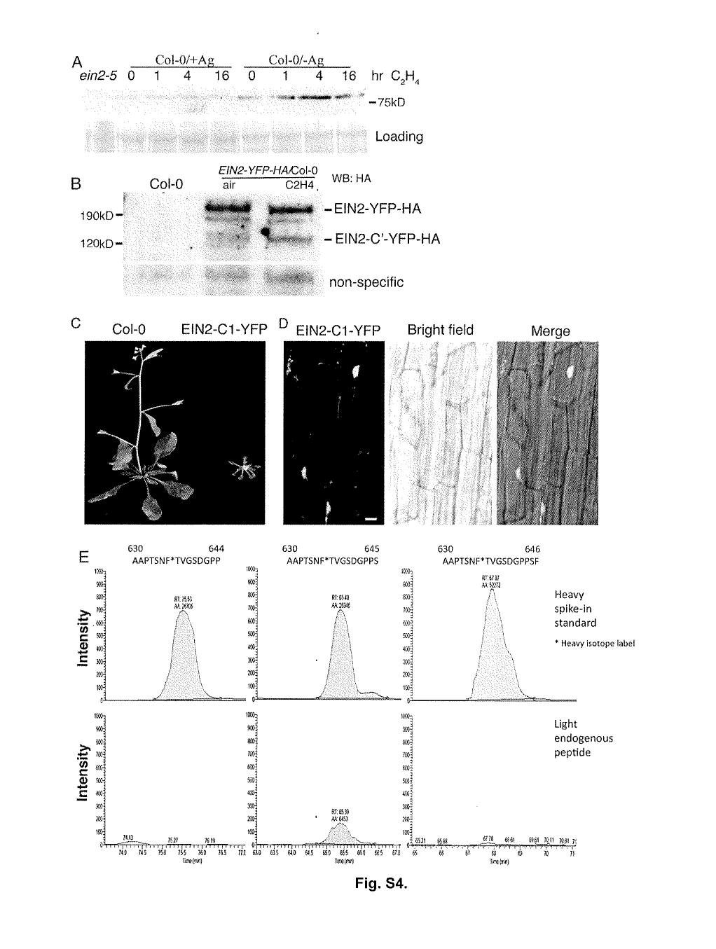

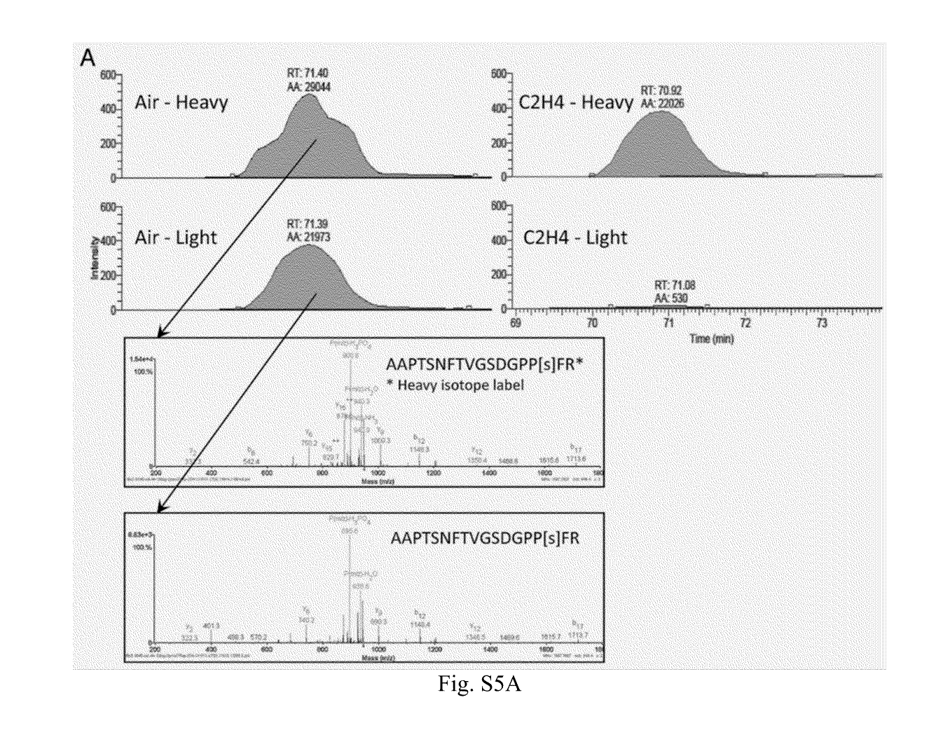

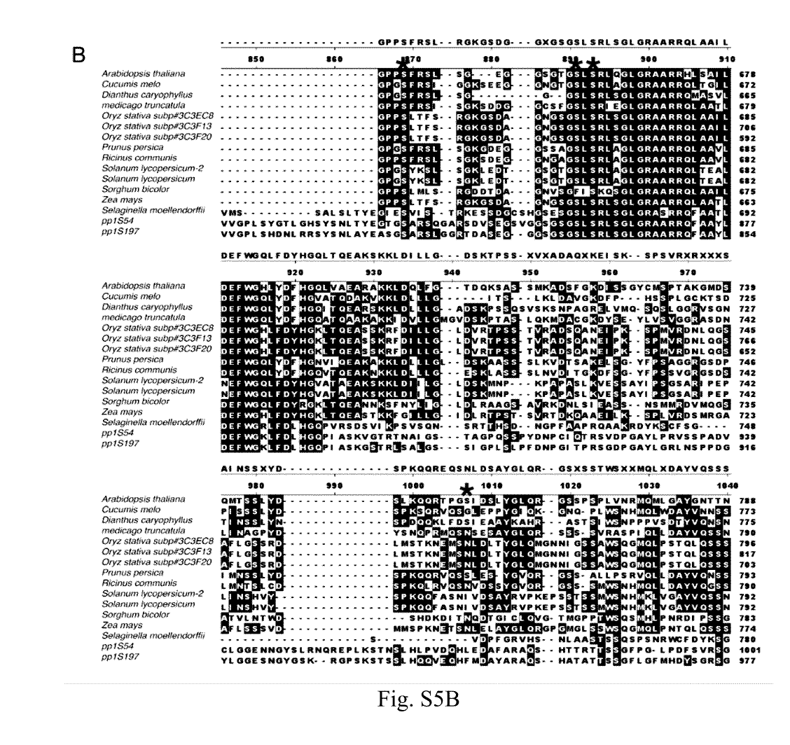

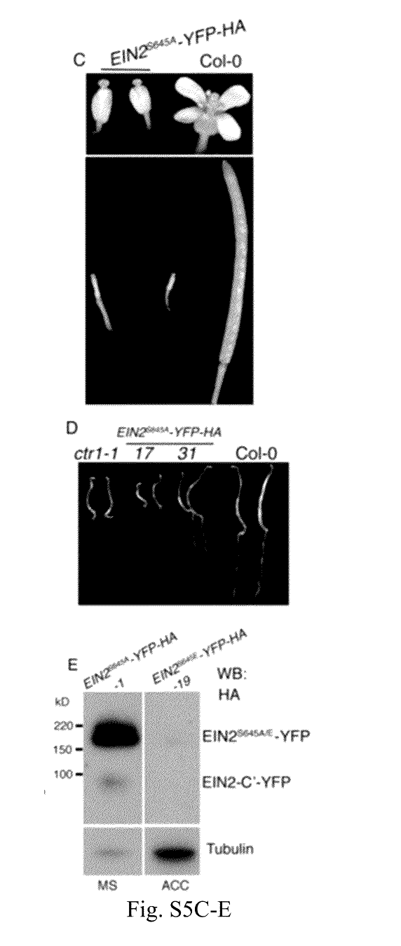

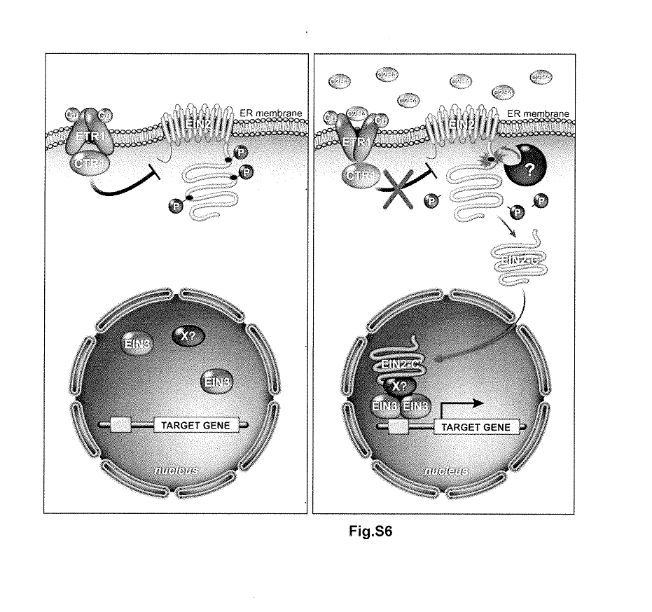

Applicants carried out a phosphoproteomic survey using proteins purified from etiolated seedlings treated with air or ethylene gas (10 ppm) and identified three sites in the EIN2 protein where phosphorylation was enriched in air-treated samples (FIG. 4A, table S1A and FIG. S5A). 5645 (SEQ ID NO:1) is conserved in all plant species examined (FIG. S5B) and this position coincided with the experimentally determined cleavage site. Applicants examined the phosphorylation status of EIN2 S645 using ctr1-1 and wild-type plants treated with or without ethylene. In wild-type plants treated with air, residue S645 of EIN2 was phosphorylated, whereas in wild-type plants treated with ethylene, S645 was not phosphorylated (FIG. 4B). In ctr1-1 mutants, however, phosphorylation of S645 was undetectable (FIG. 4B) indicating that CTRL is required for EIN2 S645 phosphorylation.

To test whether phosphorylation of S645 (SEQ ID NO:1) regulates ethylene-dependent EIN2 cleavage, constructs carrying point mutations in EIN2 that convert serine to alanine (S645A) (EIN2^{S645A} YFP-HA) or serine to glutamic acid (S645E) (EIN2^{S645E}-YFP-HA) were introduced into both wild type and ein2-5. EIN2^{S645A} did not alter the function of the EIN2 protein as it fully rescued ein2-5 in contrast to EIN2^{S645E} (FIG. 4C). In fact, etiolated seedlings and adult EIN2^{S645A} plants exhibited constitutive ethylene response phenotypes in the absence of ethylene (FIGS. 4C and 4D, FIG. S5C to S5E).

Transcriptome analysis revealed >60% of genes with significant changes in expression in EIN2^{S645A}-YFP-HA transgenic lines significantly overlapped with genes differentially expressed in wild-type ethylene treated plants (P<10^{-e200} using Fisher's exact test) or genes differentially expressed in ctr1-1 mutant plants treated with hydrocarbon-free air (FIG. 4E), suggesting that the EIN2 S645A mutation affects numerous ethylene responsive genes at the transcriptional level. Moreover, EIN2^{S645A}-YFP-HA transgenic plants showed both constitutive cleavage of EIN2 at residue 5645 and constitutive nuclear translocation of the EIN2-C′ protein (FIGS. 4F and 4G). The predicted length of the EIN2-derived polypeptide released from EIN2^{S645A}-YFP-HA matched with that observed in the nucleus of EIN2-YFP-HA transgenic plants after exposure to ethylene (FIG. 4H). In contrast, in transgenic plants containing the S645E (SEQ ID NO:1) mutant, both cleavage and nuclear translocation of EIN2-C′ protein were abolished, even in the presence of ACC (FIGS. 4F and 4G).

Applicants have uncovered a novel mechanism whereby EIN2 protein processing and subcellular nuclear translocation is required for response to ethylene (FIG. S6). Recent studies in animals have also demonstrated the importance of dephosphorylation-dependent nuclear translocation of TFEB (transcription factor EB) in regulating homeostasis of the lysosome (19), and nuclear translocation of ATFS-1 (Activating Transcription Factor associated with Stress-1) in response to mitochondrial stress (20). Further studies to determine the biochemical mechanisms that are needed for cleavage of EIN2 in response to ethylene will be of great importance. In addition, identification of the kinase(s) and phosphatase(s) that target the EIN2 protein directly as well as the enzymes that promote processing of this key regulatory molecule will be significant future challenges that will further Applicants' understanding of this highly conserved and agriculturally important plant stress and growth controlling signaling pathway.

### 5. Experimental Procedures

Plant Materials

Wild-type and mutants Arabidopsis thaliana plants used in this study (ein2-5, etr1-1, ctr1-1, ein3-1eil1-1) are in the Columbia (Col-0) background and have been previously described (1, 7, 10, 14).

Plant Growth Conditions and Hypocotyl Measurements

Arabidopsis seeds were surface-sterilized in 50% bleach with 0.01% Triton X-100 for 15 minutes and washed five times with sterile ddH2O before plating on MS medium (4.3 g MS salt, 10 g sucrose pH 5.7, 8 g phytoagar per liter) with or without addition of 10 !M ACC (Sigma). After 3-4 days of cold (4° C.) treatment, the plates were wrapped in foil and kept in at 24° C. in an incubator before the phenotypes of seedlings were analyzed. For propagation, seedlings were transferred from plates to soil (Pro-mix-HP) and grown to maturity at 22° C. under a 16 hr light/8 hr dark cycles. Ethylene treatment of Arabidopsis seedlings was performed by growth on MS plates in air-tight containers in the dark and flowing hydrocarbon-free air supplied with 10 parts per million (ppm) ethylene or hydrocarbon-free air (Zero grade air, AirGas) (7). For hypocotyl length measurements, 3-day-old seedlings were scanned using an Epson Perfection V700 Photo scanner, and hypocotyls were measured using NIH Image (On the Worldwie Web at www.rsb.info.nih.gov/nihimage).

Whole-Mount Immunofluorescent Labeling and Confocal Microscopy

Whole-mount immunofluorescence labeling was performed as described previously with minor modifications (21). Plant tissues were preserved by fixation (21). EIN2 and ACA2 were immuno-localized using an anti-EIN2 antibody and an anti-ACA2 antibody respectively, and were detected by staining with an Alexa Fluor 585 goat anti-rabbit IgG (Invitrogen). Confocal microscopy was performed using a Leica TCS SP2 AOBS confocal laser scanning microscope and an HCX PL APO 40X 1.2-numerical-aperture oil-immersion objective lens (Leica Microsystems, Mannheim, Germany). Seedlings were mounted in ddH2O. EYFP fluorescence was monitored using a 520 nm-540 nm bandpass emission and 514 nm excitation line of an Ar laser, and ECFP was monitored using a 462 nm-482 nm bandpass emission and 458 nm excitation. DAPI was detected using a 420 nm-480 nm bandpass emission and 405 nm excitation line of a Diode laser. Alexa Fluor 585 was detected using a 570 nm-650 nm bandpass emission and 561 nm excitation line of a Diode laser.

Plant Protein Extraction

Arabidopsis seedlings grown in the indicated conditions were harvested and immediately frozen in liquid N2 and stored at −80° C. until processing. For total plant protein extraction, frozen seedlings were ground in liquid N2 and mixed with extraction buffer (100 mM Tris-HCl pH 7.5, 100 mM NaCl, 5 mM EDTA, 10 mM N-ethylmaleimide, 5 mM DTT, 10 mM β-mercaptoethanol and 1% SDS, and protease inhibitors from Sigma P8465), and centrifuged at 10,000 # g for 10 min at 4° C. The supernatant was collected for further analysis.

Sucrose Density-Gradient Centrifugation

Sucrose density-gradient centrifugation was performed by fractionation of microsomal membranes containing Mg²⁺ to stabilize membrane-associated proteins, or in the absence of Mg²⁺, to dissociate membrane-associated proteins. Briefly, total membrane fractions were extracted as previously described (13) and the homogenized samples were subjected to centrifugation in a 5-40% sucrose gradient at 190,000×g (37500) rpm for 16 hours using a SW55Ti rotor. Collection of density gradient fractions (500 ul) was followed by western blot analysis.

Western Blotting Analysis

Proteins were resolved by SDS-PAGE and electroblotted onto a nitrocellulose membrane and probed with the indicated primary antibodies and then with secondary goat anti-rabbit (Bio-rad 170-6515) or goat anti-mouse (Bio-rad 170-6516) antibodies conjugated with horseradish peroxidase. The signals were detected by a chemiluminescence reaction using the SuperSignal® kit (Pierce). Polyclonal anti-GFP antibodies (Invitrogen) were used at dilution of 1:1000. Polyclonal anti-EIN2 antibodies were used at dilution of 1:4000. Polyclonal anti-ACA2 antibodies were used at dilution of 1:4000. Polyclonal anti-histone H3 (BioMol) was used at dilution of 1:5000. Monoclonal anti-HA (Cell signaling) was used at dilution of 1:5000.

Gene Expression Analysis by Quantitative Real-Time PCR

Total RNA was extracted using a Qiagen Plant Total RNA Kit (Sigma) from 7-week-old Col-0 seedlings grown in MS media provided with or without 20!M DEX. First strand cDNA was synthesized using Invitrogen Superscript III First-Strand cDNA Synthesis Kit. cDNAs were combined with SYBR master mix from BIOLINE for PCR. Primers for ERF1 are: GAGGATGGTTGTTCTCCGGTG (SEQ ID NO:19) and ACGGAGCGGTGATCAAAGTCA (SEQ ID NO:20). Primers for PDF1.2 are: CGTTCAGCATCTGGAGTTTCAC (SEQ ID NO:21) and CCATCATCACCCTTATCTTCG (SEQ ID NO:22). PCR reactions were performed in triplicate with an Eppendorf Mastercycler ep realplex Thermal cycler.

GUS Staining

GUS staining was performed using minor modifications of a previously described method (22). Briefly, seedlings were fixed in 90% acetone on ice for 20 min, rinsed with staining solution (50 mM sodium phosphate buffer pH 7.2, 0.2% Triton X-100, 10 mM potassium ferrocyanide, 10 mM potassium ferricyanide, and 1 mM X-gluc), vacuum infiltrated with the staining solution for 15 minutes, and incubated at 37° C. for 12 hours. Samples were dehydrated through 30 minutes incubation of 20% ethanol, 35% ethanol, 50% ethanol, and fixed in FAA for 30 minutes at room temperature. Samples were then washed in 70% ethanol for 30 minutes and 30% glycerol for 1 hour before mounted on slides in 30% glycerol.

Purification of Nuclei and Membrane Fractionation

Membrane and nuclei fractions from 3-day-old dark-grown Col-0 seedlings treated with or without ethylene were prepared as follows. Membrane fractionation was carried out by a protocol described previously (13). One gram of Arabidopsis tissue was homogenized with 2 ml of cold homogenization buffer (30 mM Tris, pH7.4, 150 mM NaCl, 10 mM EDTA, 20% Glycerol with proteins inhibitor cocktail from Sigma). The homogenate was filtered through two layers of miracloth and centrifuged for 5 minutes at 10,000 g to spin down debris and organelles. The supernatants were centrifuged 30 minutes at 100,000 g to pellet the membrane fraction. To isolate the nuclear fraction, one gram of Arabidopsis tissue was homogenized gently using a polytron tissue homogenizor and the tissue was suspended in 0.75 ml of extraction buffer (20 mM PIPES-KOH pH 7.0, 4M hexylene glycol, 10 mM MgCl2, 0.25% Triton X-100, 4 mM 2-mercaptoethanol, and complete miniprotease inhibitor cocktail from Roche). The crude extract was filtered through miracloth and passed through 800 ul of 30% and 80% percoll gradient by centrifugation at 2000 g for 30 minutes, the nuclei banded at the 30-80% interface. Protein extracts from membrane and nuclear fractions were resolved by SDS-PAGE, and EIN2 was detected by western blotting using anti-EIN2 antibodies. Calmodulin-stimulated Ca²⁺ Pump (ACA2) and histone H3 were used as controls to monitor the purity of the membrane and nuclear fractions. The anti-ACA2 antibody was kindly provided by J Harper (University of Nevada Reno) and the anti-histone H3 antibody was obtained from Cell Signaling (Cambridge, Mass.).

Mass Spectrometry

For Arabidopsis seedling samples, frozen tissues were ground in liquid nitrogen for 15 minutes to a fine powder, then transferred to a 50 ml conical tube. Samples were washed by 50 ml −20° C. methanol with 0.2 mM Na3VO4 three times, then by 50 ml −20° C. acetone three times. Protein pellets were dried in a SpeedVac at 4° C. Proteins were extracted by adding 0.2% RapiGest (Waters) in 50 mM Hepes (pH 7.2) with 0.2 mM Na3VO4 to the dry pellet. For membrane samples, proteins were extracted by adding 0.2% RapiGest (Waters) in 50 mM Hepes (pH 7.2) with 0.2 mM Na3VO4 to the dry pellet. Cysteines were reduced and alkylated using 1 mM Tris(2-carboxyethyl)phosphine (Fisher, AC36383) at 95° C. for 5 minutes then 2.5 mM iodoacetamide (Fisher, AC12227) at 37° C. in dark for 15 minutes. Proteins were digested with trypsin (Roche, 03 708 969 001, enzyme:substrate w:w ratio=1:50) overnight then 1% TFA (pH 1.4) was added to precipitate RapiGest. Samples were incubated at 4° C. overnight and then centrifuged at 16,100 g for 15 minutes. Supernatant was collected and centrifuged through a 0.22 uM filter.

For global phosphoproteome profiling, phosphopeptides were enriched by metal oxide (CeO2) affinity method. For pseudo-MRM experiments, heavy labeled synthetic peptides (Thermo) were spiked into the samples right after trypsin digestion, before the RapiGest removal step to minimize quantitation error.

Automated 2D nanoflow LC-MS/MS analysis was performed using LTQ tandem mass spectrometer (Thermo Electron Corporation, San Jose, Calif.) employing automated data-dependent acquisition. An Agilent 1100 HPLC system (Agilent Technologies, Wilmington, Del.) was used to deliver a flow rate of 500 nL/min to the mass spectrometer through a splitter. Chromatographic separation was accomplished using a 3 phase capillary column. Using an in-house constructed pressure cell, Sum Zorbax SB-C18 (Agilent) packing material was packed into a fused silica capillary tubing (200!m ID, 360 um OD, 10 cm long) to form the first dimension RP column (RP1). A similar column (200m ID, 5 cm long) packed with 5 um PolySulfoethyl (PolyLC) packing material was used as the SCX column. A zero dead volume 1!m filter (Upchurch, M548) was attached to the exit of each column for column packing and connecting. A fused silica capillary (200!m ID, 360 um OD, 20 cm long) packed with 5 um Zorbax SB-C18 (Agilent) packing material was used as the analytical column (RP2). One end of the fused silica tubing was pulled to a sharp tip with the ID smaller than 1!m using a laser puller (Sutter P-2000) as the electro-spray tip. The peptide mixtures were loaded onto the RP1 column using the same in-house pressure cell. To avoid sample carry-over and maintain high reproducibility, a new set of three columns with the same length was used for each sample. Peptides were first eluted from RP1 column to SCX column using a 0 to 80% acetonitrile gradient for 150 minutes.

For global phosphoproteome profiling experiments, peptides were fractionated by the SCX column using a series of 19 step salt gradients (5 mM, 6 mM, 7 mM, 8 mM, 9 mM, 10 mM, 12 mM, 15 mM, 20 mM, 30 mM, 40 mM, 50 mM, 60 mM, 70 mM, 80 mM, 90 mM, 100 mM, and 1M ammonium acetate for 20 minutes), followed by high resolution reverse phase separation using an acetonitrile gradient of 0 to 80% for 120 minutes. Data-dependent analysis and gas phase separation were employed. The full MS scan range of 300-2000 m/z was divided into 3 smaller scan ranges (300-800, 800-1100, 1100-2000 Da) to improve the dynamic range. Each MS scan was followed by 4 MS/MS scans of the most intense ions from the parent MS scan. A dynamic exclusion of 1 minute was used to improve the duty cycle of MS/MS scans. About 500,000 MS/MS spectra were collected for each run.

For pseudo-MRM runs, mass spectrometer was programmed to perform data-independent MS/MS scans on the peptides of interest. The MS/MS scans of the heavy/light peptide pairs are always acquired right next to each other. Dynamic exclusion was not used here. Raw data were extracted and searched using Spectrum Mill (Agilent, version B04.00). MS/MS spectra with a sequence tag length of 1 or less were considered as poor spectra and discarded. The filtered of the MS/MS spectra were searched against the IPI (International Protein Index) database limited to Arabidopsis taxonomy v3.29 (July, 2007) (35,619 protein sequences). The enzyme parameter was limited to full tryptic peptides with a maximum miscleavage of 2. All other search parameters were set to SpectrumMill's default settings (carbamidomethylation of cysteines, +/−2.5 Da for precursor ions, +/−0.7 Da for fragment ions, and a minimum matched peak intensity of 50%). Ox-Met, n-term pyro-Gln, and phosphorylation on Serine, Threonine, or Tyrosine were defined as variable modifications for phosphoproteome data. A maximum of 2 modifications per peptide was used. A 1:1 concatenated forward-reverse database was constructed to calculate the false discovery rate (FDR). The tryptic peptides in the reverse database were compared to the forward database, and were shuffled if they matched to any tryptic peptides from the forward database. The total number of protein sequences in the concatenated database is 71,238. Peptide cutoff scores were dynamically assigned to each dataset to maintain the false discovery rate (FDR) less than 1% at the peptide level. Proteins that share common peptides were grouped to address the protein database redundancy issue. The proteins within the same group shared the same set or subset of unique peptides. A total of 3,528 phosphopeptides from 1,186 protein groups were identified. The cutoff scores were 11.8, 13.0, and 16.1 for singly, doubly and triply charged peptides, respectively. The FDR of the entire phosphoproteome profiling dataset were 1.6% at the unique phosphopeptide level, and 1.9% at the phosphoprotein group level, respectively.

Gene Expression Experiments

RNA for wild type, ctr-1-1 and EIN2^{S645A}-YFP-HA transgenic lines treated with ethylene gas or hydrocarbon-free air were isolated following the manufacturer's recommendation in the RNeasy Plant Kit (Qiagen, CA). cDNA sequencing libraries were prepared according to the instructions include in the Illumina TruSeq v2 library preparation kit (Illumina, CA) Reads were mapped using TopHat, and analyzed using Cufflinks according to Trapnell et al., 2012 (23). Reads were mapped to the TAIR 10 genome assembly using TopHat, and analyzed using Cufflinks. Differentially expressed genes were identified by fragments per kilobase per million reads (FPKM) filter<0.1, requiring a 2-fold change comparing the indicated conditions with P<=0.05 after Benjamin-Hochberg correction.

### 6. Tables

TABLE S1A
Absolute quantitation of EIN2 peptides by Pseudo-MRM.

| | | | Amount | Amount | |
|---|---|---|---|---|---|
| Peptide | Precurso | Fragment | Col-0 -Air | Col-0 —C₂H₄ | Air/C₂H₄ |
| [S]: phosphorylation site | r m/z (2+) | ion, m/z | (pmol/100 ug) | (pmol/100 ug) | Ratio |
| 630 AAPTSNFTVGSDGPPSFR | 904.4 | y11+, 1119.5 | 0.0638 | 0.0033 | 19.2 |
| 647 | | | | | |
| 630 AAPTSNFTVGSDGPP[S]FR | 944.4 | y9+, 999.4 | 0.0685 | 0.0012 | 55.3 |
| 647 | | | | | |
| 648 SLSGEGGSGTGSLSR 662 | 676.3 | y10+, 878.4 | 0.0060 | 0.0120 | 0.5 |
| 648 SLSGEGGSGTG[S]LSR 662 | 716.3 | y10+, 958.4 | 0.0197 | 0.0020 | 9.8 |
| 754 TPGSIDSLYGLQR 766 | 703.9 | y8+, 951.5 | 0.0483 | 0.0255 | 1.9 |
| 754 TPG[s]IDSLYGLQR 766 | 743.9 | y8+, 951.5 | 0.0058 | Not detectable | |

TABLE S1B
18 synthetic heavy isotope labeled
peptides used for mapping the EIN2
cleavage site. Heavy Arg (+10Da)
isotope composition: 13C6, 99%;
15N4, 99%. Heavy Phe (+10Da) isotope
composition: 13C9, 99%; 15N1, 99%.

| | | | Endogenous |
|---|---|---|---|
| | | | light peptide/ |
| | Start | End | spike-in |
| *:Heavy Isotope Labeled | AA | AA | heavy peptide |
| AAPTSNFTVGSDGPPSFR* | 630 | 647 | N/D |
| APTSNFTVGSDGPPSFR* | 631 | 647 | N/D |
| PTSNFTVGSDGPPSFW | 632 | 647 | N/D |
| TSNFTVGSDGPPSFR* | 633 | 647 | N/D |
| SNFTVGSDGPPSFR* | 634 | 647 | N/D |
| NFTVGSDGPPSFR* | 635 | 647 | N/D |
| FTVGSDGPPSFR* | 636 | 647 | N/D |
| TVGSDGPPSFR* | 637 | 647 | N/D |
| VGSDGPPSFR* | 638 | 647 | N/D |
| GSDGPPSFR* | 639 | 647 | N/D |
| AAPTSNF*TVG | 630 | 639 | Refer to Fig3C |
| AAPTSNF*TVGS | 630 | 640 | Refer to Fig3C |
| AAPTSNF*TVGSD | 630 | 641 | Refer to Fig3C |
| AAPTSNF*TVGSDG | 630 | 642 | Refer to Fig3C |
| AAPTSNF*TVGSDGP | 630 | 643 | Refer to Fig3C |
| AAPTSNF*TVGSDGPP | 630 | 644 | Refer to Fig3C |
| AAPTSNF*TVGSDGPPS | 630 | 645 | Refer to Fig3C |
| AAPTSNF*TVGSDGPPSF | 630 | 646 | Refer to Fig3C |

N/D = not detectable.

### 7. References

1. C. Chang, S. F. Kwok, A. B. Bleecker, E. M. Meyerowitz, Arabidopsis ethylene-response gene ETR1: similarity of product to two-component regulators. Science 262, 539 (Oct. 22, 1993).
2. J. Hua, E. M. Meyerowitz, Ethylene responses are negatively regulated by a receptor gene family in Arabidopsis thaliana. Cell 94, 261 (Jul. 24, 1998).
3. H. Sakai et al., ETR2 is an ETR1-like gene involved in ethylene signaling in Arabidopsis. Proc Natl Acad Sci USA 95, 5812 (May 12, 1998).
4. Y. F. Chen et al., Ethylene receptors function as components of high-molecular-mass protein complexes in Arabidopsis. PloS one 5, e8640 (2010).
5. T. Hirayama et al., RESPONSIVE-TO-ANTAGONIST1, a Menkes/Wilson disease-related copper transporter, is required for ethylene signaling in Arabidopsis. Cell 97, 383 (Apr. 30, 1999).
6. C. H. Dong et al., Molecular association of the Arabidopsis ETR1 ethylene receptor and a regulator of ethylene signaling, RTE1. J Biol Chem 285, 40706 (Dec. 24, 2010).
7. J. J. Kieber, M. Rothenberg, G. Roman, K. A. Feldmann, J. R. Ecker, CTR1, a negative regulator of the ethylene response pathway in Arabidopsis, encodes a member of the raf family of protein kinases. Cell 72, 427 (Feb. 12, 1993).
8. Y. Huang, H. Li, C. E. Hutchison, J. Laskey, J. J. Kieber, Biochemical and functional analysis of CTR1, a protein kinase that negatively regulates ethylene signaling in Arabidopsis. Plant J 33, 221 (January, 2003).
9. M. D. Kendrick, C. Chang, Ethylene signaling: new levels of complexity and regulation. Curr Opin Plant Biol 11, 479 (October, 2008).
10. G. Roman, B. Lubarsky, J. J. Kieber, M. Rothenberg, J. R. Ecker, Genetic analysis of ethylene signal transduction in Arabidopsis thaliana: five novel mutant loci integrated into a stress response pathway. Genetics 139, 1393 (March, 1995).
11. J. M. Alonso, T. Hirayama, G. Roman, S. Nourizadeh, J. R. Ecker, EIN2, a bifunctional transducer of ethylene and stress responses in Arabidopsis. Science 284, 2148 (Jun. 25, 1999).
12. M. M. Bisson, A. Bleckmann, S. Allekotte, G. Groth, EIN2, the central regulator of ethylene signalling, is localized at the ER membrane where it interacts with the ethylene receptor ETR1. Biochem J 424, 1 (Nov. 15, 2009).
13. H. Qiao, K. N. Chang, J. Yazaki, J. R. Ecker, Interplay between ethylene, ETP1/ETP2 F-box proteins, and degradation of EIN2 triggers ethylene responses in Arabidopsis. Genes Dev 23, 512 (Feb. 15, 2009).
14. Q. Chao et al., Activation of the ethylene gas response pathway in Arabidopsis by the nuclear protein ETHYLENE-INSENSITIVE3 and related proteins. Cell 89, 1133 (Jun. 27, 1997).
15. M. M. Bisson, G. Groth, New paradigm in ethylene signaling: EIN2, the central regulator of the signaling pathway, interacts directly with the upstream receptors. Plant signaling & behavior 6, 164 (January, 2011).
16. B. M. Binder, L. A. Mortimore, A. N. Stepanova, J. R. Ecker, A. B. Bleecker, Short-term growth responses to ethylene in Arabidopsis seedlings are EIN3/EIL1 independent. Plant Physiol 136, 2921 (October, 2004).
17. B. F. Cravatt, G. M. Simon, J. R. Yates, 3rd, The biological impact of mass-spectrometry-based proteomics. Nature 450, 991 (Dec. 13, 2007).
18. V. Lange, P. Picotti, B. Domon, R. Aebersold, Selected reaction monitoring for quantitative proteomics: a tutorial. Molecular systems biology 4, 222 (2008).
19. A. Roczniak-Ferguson et al., The Transcription Factor TFEB Links mTORC1 Signaling to Transcriptional Control of Lysosome Homeostasis. Science signaling 5, ra42 (2012).
20. A. M. Nargund, M. W. Pellegrino, C. J. Fiorese, B. M. Baker, C. M. Haynes, Mitochondrial Import Efficiency of ATFS-1 Regulates Mitochondrial UPR Activation. Science, (Jun. 14, 2012).
21. M. Sauer, T. Paciorek, E. Benkova, J. Friml, Immunocytochemical techniques for whole-mount in situ protein localization in plants. Nature protocols 1, 98 (2006).
22. R. A. Jefferson, T. A. Kavanagh, M. W. Bevan, GUS fusions: beta-glucuronidase as a sensitive and versatile gene fusion marker in higher plants. EMBO J 6, 3901 (Dec. 20, 1987).
23. C. Trapnell et al., Differential gene and transcript expression analysis of RNA-seq experiments with TopHat and Cufflinks. Nature protocols 7, 562 (March, 2012).
24. T. A. Haq, H. S. Mason, J. D. Clements, C. J. Arntzen, Oral immunization with a recombinant bacterial antigen produced in transgenic plants. Science 268, 714 (May 5, 1995).
25. Q. Zhao, H. W. Guo, Paradigms and paradox in the ethylene signaling pathway and interaction network. Molecular plant 4, 626 (July, 2011).

### 8. Embodiments

### Embodiment 1

A non-naturally occurring plant expressing an EIN2 protein comprising an amino acid mutation at a position corresponding to position 645 of SEQ ID NO:1, wherein said non-naturally occurring plant has modulated ethylene sensitivity compared to a wildtype plant.

### Embodiment 2

The non-naturally occurring plant of embodiment 1, wherein said amino acid mutation mimics an unphosphorylated serine.

### Embodiment 3

The non-naturally occurring plant of embodiment 2, wherein said amino acid mutation is a serine to alanine mutation.

### Embodiment 4

The non-naturally occurring plant of embodiment 3, wherein expressing said EIN2 protein increases ethylene sensitivity of said non-naturally occurring plant compared to a wildtype plant.

### Embodiment 5

The non-naturally occurring plant of embodiment 1, wherein said amino acid mutation mimics a phosphorylated serine.

### Embodiment 6

The non-naturally occurring plant of embodiment 5, wherein said amino acid mutation is a serine to glutamic acid mutation.

### Embodiment 7

The non-naturally occurring plant of embodiment 6, wherein expressing said EIN2 protein decreases ethylene sensitivity of said non-naturally occurring plant compared to a wildtype plant.

### Embodiment 8

The non-naturally occurring plant of embodiment 1, wherein said EIN2 protein is encoded by a nucleic acid operably linked to an inducible promoter.

### Embodiment 9

The non-naturally occurring plant of embodiment 1, wherein said EIN2 protein is encoded by a nucleic acid operably linked to a tissue-specific promoter.

### Embodiment 10

The non-naturally occurring plant of embodiment 1, wherein said EIN2 protein is encoded by a nucleic acid operably linked to an endogenous promoter or an exogenous promoter.

### Embodiment 11

The non-naturally occurring plant of embodiment 1, wherein said plant is a transgenic plant.

### Embodiment 12

The non-naturally occurring plant of embodiment 1, wherein said plant is selected from the group consisting of rice, maize, wheat, barley, sorghum, millet, grass, moss, oats, tomato, potato, legume, banana, kiwi fruit, avocado, melon, mango, cane, sugar beet, tobacco, papaya, peach, strawberry, raspberry, blackberry, blueberry, lettuce, cabbage, cauliflower, onion, broccoli, brussels sprouts, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, castor oil plant, spinach, apples, cherries, cranberries, grapefruit, lemons, limes, nectarines, oranges, pears, tangelos, tangerines, lily, carnation, chrysanthemum, petunia, rose, geranium, violet, gladioli, orchid, lilac, crabapple, sweetgum, maple, poinsettia, locust, ash, linden tree, poplar tree and Arabidopsis thaliana.

### Embodiment 13

The non-naturally occurring plant of embodiment 1, wherein said plant is selected from the group consisting of Arabidopsis thaliana, melon, legume, rice, petunia, poplar tree, peach and tomato.

### Embodiment 14

The non-naturally occurring plant of embodiment 1, wherein said plant is selected from the group consisting of Arabidopsis thaliana, melon, carnation, legume, peach, castor oil plant, tomato, sorghum, corn and selaginella.

### Embodiment 15

A non-naturally occurring plant expressing an EIN2 protein comprising a serine to alanine mutation at a position corresponding to position 645 of SEQ ID NO:1.

### Embodiment 16

The non-naturally occurring plant of embodiment 15, wherein said plant is selected from the group consisting of rice, maize, wheat, barley, sorghum, millet, grass, moss, oats, tomato, potato, legume, banana, kiwi fruit, avocado, melon, mango, cane, sugar beet, tobacco, papaya, peach, strawberry, raspberry, blackberry, blueberry, lettuce, cabbage, cauliflower, onion, broccoli, brussels sprouts, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, castor oil plant, spinach, apples, cherries, cranberries, grapefruit, lemons, limes, nectarines, oranges, pears, tangelos, tangerines, lily, carnation, chrysanthemum, petunia, rose, geranium, violet, gladioli, orchid, lilac, crabapple, sweetgum, maple, poinsettia, locust, ash, linden tree, poplar tree and Arabidopsis thaliana.

### Embodiment 17

The non-naturally occurring plant of embodiment 15, wherein said plant is selected from the group consisting of Arabidopsis thaliana, melon, legume, rice, petunia, poplar tree, peach and tomato.

### Embodiment 18

The non-naturally occurring plant of embodiment 15, wherein said plant is selected from the group consisting of Arabidopsis thaliana, melon, carnation, legume, peach, castor oil plant, tomato, sorghum, corn and selaginella.

### Embodiment 19

A non-naturally occurring plant expressing an EIN2 protein comprising a serine to glutamic acid mutation at a position corresponding to position 645 of SEQ ID NO:1.

### Embodiment 20

The non-naturally occurring plant of embodiment 19, wherein said plant is selected from the group consisting of rice, maize, wheat, barley, sorghum, millet, grass, moss, oats, tomato, potato, legume, banana, kiwi fruit, avocado, melon, mango, cane, sugar beet, tobacco, papaya, peach, strawberry, raspberry, blackberry, blueberry, lettuce, cabbage, cauliflower, onion, broccoli, brussels sprouts, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, castor oil plant, spinach, apples, cherries, cranberries, grapefruit, lemons, limes, nectarines, oranges, pears, tangelos, tangerines, lily, carnation, chrysanthemum, petunia, rose, geranium, violet, gladioli, orchid, lilac, crabapple, sweetgum, maple, poinsettia, locust, ash, linden tree, poplar tree and Arabidopsis thaliana.

### Embodiment 21

The non-naturally occurring plant of embodiment 19, wherein said plant is selected from the group consisting of Arabidopsis thaliana, melon, legume, rice, petunia, poplar tree, peach and tomato.

### Embodiment 22

The non-naturally occurring plant of embodiment 19, wherein said plant is selected from the group consisting of Arabidopsis thaliana, melon, carnation, legume, peach, castor oil plant, tomato, sorghum, corn and selaginella.

### Embodiment 23

A recombinant expression cassette comprising a promoter operably linked to a nucleic acid encoding an EIN2 protein, wherein said EIN2 protein comprises an amino acid mutation at a position corresponding to position 645 of SEQ ID NO:1.

### Embodiment 24

The recombinant expression cassette of embodiment 23, wherein said amino acid mutation mimics an unphosphorylated serine.

### Embodiment 25

The recombinant expression cassette of embodiment 24, wherein said amino acid mutation is a serine to alanine mutation.

### Embodiment 26

The recombinant expression cassette of embodiment 25, wherein said EIN2 protein increases ethylene sensitivity in a plant expressing said recombinant expression cassette compared to a control plant lacking said expression cassette.

### Embodiment 27

The recombinant expression cassette of embodiment 23, wherein said amino acid mutation mimics a phosphorylated serine.

### Embodiment 28

The recombinant expression cassette of embodiment 27, wherein said amino acid mutation is a serine to glutamic acid mutation.

### Embodiment 29

The recombinant expression cassette of embodiment 28, wherein said EIN2 protein decreases ethylene sensitivity in a plant expressing said recombinant expression cassette compared to a control plant lacking said expression cassette.

### Embodiment 30

The recombinant expression cassette of embodiment 23, wherein said promoter is an inducible promoter.

### Embodiment 31

The recombinant expression cassette of embodiment 23, wherein said promoter is a tissue-specific promoter.

### Embodiment 32

The recombinant expression cassette of embodiment 23, wherein said promoter is an endogenous promoter or an exogenous promoter.

### Embodiment 33

A recombinant nucleic acid encoding an EIN2 protein comprising a serine to alanine mutation at a position corresponding to position 645 of SEQ ID NO:1.

### Embodiment 34

A recombinant nucleic acid encoding an EIN2 protein comprising a serine to glutamic acid mutation at a position corresponding to position 645 of SEQ ID NO:1.

### Embodiment 35

A method of making a plant of any one of embodiments 1-22, the method comprising introducing a nucleic acid encoding an EIN2 protein comprising an amino acid mutation at a position corresponding to position 645 of SEQ ID NO:1 into a plurality of plants; and selecting a plant that expresses said EIN2 protein from the plurality of plants.

### Embodiment 36

The method of embodiment 35, wherein the selecting step comprises selecting a plant that has altered ethylene sensitivity.

### Embodiment 37

The method of embodiment 35, wherein said amino acid mutation is a serine to alanine mutation.

### Embodiment 38

The method of embodiment 35, wherein said amino acid mutation is a serine to glutamic acid mutation.

1. A non-naturally occurring plant expressing an EIN2 protein comprising a serine to alanine mutation at a position corresponding to position 645 of SEQ ID NO:1.

2. A non-naturally occurring plant expressing an EIN2 protein comprising a serine to glutamic acid mutation at a position corresponding to position 645 of SEQ ID NO:1.

3. (canceled)

4. (canceled)

5. A non-naturally occurring plant expressing an EIN2 protein comprising an amino acid mutation at a position corresponding to position 645 of SEQ ID NO:1, wherein said non-naturally occurring plant has modulated ethylene sensitivity compared to a wildtype plant.

6. The non-naturally occurring plant of claim 5, wherein said amino acid mutation mimics an unphosphorylated serine.

7. The non-naturally occurring plant of claim 6, wherein expressing said EIN2 protein increases ethylene sensitivity of said non-naturally occurring plant compared to a wildtype plant.

8. The non-naturally occurring plant of claim 5, wherein said amino acid mutation mimics a phosphorylated serine.

9. The non-naturally occurring plant of claim 8, wherein expressing said EIN2 protein decreases ethylene sensitivity of said non-naturally occurring plant compared to a wildtype plant.

10. The non-naturally occurring plant of claim 5, wherein said plant is selected from the group consisting of rice, maize, wheat, barley, sorghum, millet, grass, moss, oats, tomato, potato, legume, banana, kiwi fruit, avocado, melon, mango, cane, sugar beet, tobacco, papaya, peach, strawberry, raspberry, blackberry, blueberry, lettuce, cabbage, cauliflower, onion, broccoli, brussels sprouts, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, castor oil plant, spinach, apples, cherries, cranberries, grapefruit, lemons, limes, nectarines, oranges, pears, tangelos, tangerines, lily, carnation, chrysanthemum, petunia, rose, geranium, violet, gladioli, orchid, lilac, crabapple, sweetgum, maple, poinsettia, locust, ash, linden tree, poplar tree and Arabidopsis thaliana.

11. The non-naturally occurring plant of claim 1, wherein said plant is selected from the group consisting of rice, maize, wheat, barley, sorghum, millet, grass, moss, oats, tomato, potato, legume, banana, kiwi fruit, avocado, melon, mango, cane, sugar beet, tobacco, papaya, peach, strawberry, raspberry, blackberry, blueberry, lettuce, cabbage, cauliflower, onion, broccoli, brussels sprouts, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, castor oil plant, spinach, apples, cherries, cranberries, grapefruit, lemons, limes, nectarines, oranges, pears, tangelos, tangerines, lily, carnation, chrysanthemum, petunia, rose, geranium, violet, gladioli, orchid, lilac, crabapple, sweetgum, maple, poinsettia, locust, ash, linden tree, poplar tree and Arabidopsis thaliana.

12. The non-naturally occurring plant of claim 2, wherein said plant is selected from the group consisting of rice, maize, wheat, barley, sorghum, millet, grass, moss, oats, tomato, potato, legume, banana, kiwi fruit, avocado, melon, mango, cane, sugar beet, tobacco, papaya, peach, strawberry, raspberry, blackberry, blueberry, lettuce, cabbage, cauliflower, onion, broccoli, brussels sprouts, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, castor oil plant, spinach, apples, cherries, cranberries, grapefruit, lemons, limes, nectarines, oranges, pears, tangelos, tangerines, lily, carnation, chrysanthemum, petunia, rose, geranium, violet, gladioli, orchid, lilac, crabapple, sweetgum, maple, poinsettia, locust, ash, linden tree, poplar tree and Arabidopsis thaliana.

13. A recombinant expression cassette comprising a promoter operably linked to a nucleic acid encoding an EIN2 protein, wherein said EIN2 protein comprises an amino acid mutation at a position corresponding to position 645 of SEQ ID NO:1.

14. The recombinant expression cassette of claim 13, wherein said amino acid mutation mimics an unphosphorylated serine.

15. The recombinant expression cassette of claim 14, wherein said EIN2 protein increases ethylene sensitivity in a plant expressing said recombinant expression cassette compared to a control plant lacking said expression cassette.

16. The recombinant expression cassette of claim 13, wherein said amino acid mutation mimics a phosphorylated serine.

17. The recombinant expression cassette of claim 16, wherein said EIN2 protein decreases ethylene sensitivity in a plant expressing said recombinant expression cassette compared to a control plant lacking said expression cassette.

18. A method of making a plant of any one of claim 1, 2, or 5, the method comprising introducing a nucleic acid encoding an EIN2 protein comprising an amino acid mutation at a position corresponding to position 645 of SEQ ID NO:1 into a plurality of plants; and selecting a plant that expresses said EIN2 protein from the plurality of plants.


SEQUENCE LISTING (22 sequences)

SEQ ID NO: 1 - protein, 1294 aa, Arabidopsis thaliana
  VARIANT (645)..(645): Residue S at position 645 may be A or E
MEAEIVNVRPQLGFIQRMVPALLPVLLVSVGYIDPGKWVANIEGGARFGYDLVAITLLFNFAAILCQYVAARISVVTGKHLAQICNEEYDKWTCMFLGIQAEFSAILLDLTMVVGVAHALNLLFGVELSTGVFLAAMDAFLFPVFASFLENGMANTVSIYSAGLVLLLYVSGVLLSQSEIPLSMNGVLTRLNGESAFALMGLLGASIVPHNFYIHSYFAGESTSSSDVDKSSLCQDHLFAIFGVFSGLSLVNYVLMNAAANVFHSTGLVVLTFHDALSLMEQVFMSPLIPVVFLMLLFFSSQITALAWAFGGEVVLHDFLKIEIPAWLHRATIRILAVAPALYCVWTSGADGIYQLLIFTQVLVAMMLPCSVIPLFRIASSRQIMGVHKIPQVGEFLALTTFLGFLGLNVVFVVEMVFGSSDWAGGLRWNTVMGTSIQYTTLLVSSCASLCLILWLAATPLKSASNRAEAQIWNMDAQNALSYPSVQEEEIERTETRRNEDESIVRLESRVKDQLDTTSVTSSVYDLPENILMTDQEIRSSPPEERELDVKYSTSQVSSLKEDSDVKEQSVLQSTVVNEVSDKDLIVETKMAKIEPMSPVEKIVSMENNSKFIEKDVEGVSWETEEATKAAPTSNFTVGSDGPPSFRSLSGEGGSGTGSLSRLQGLGRAARRHLSAILDEFWGHLYDFHGQLVAEARAKKLDQLFGTDQKSASSMKADSFGKDISSGYCMSPTAKGMDSQMTSSLYDSLKQQRTPGSIDSLYGLQRGSSPSPLVNRMQMLGAYGNTTNNNNAYELSERRYSSLRAPSSSEGWEHQQPATVHGYQMKSYVDNLAKERLEALQSRGEIPTSRSMALGTLSYTQQLALALKQKSQNGLTPGPAPGFENFAGSRSISRQSERSYYGVPSSGNTDTVGAAVANEKKYSSMPDISGLSMSARNMHLPNNKSGYWDPSSGGGGYGASYGRLSNESSLYSNLGSRVGVPSTYDDISQSRGGYRDAYSLPQSATTGTGSLWSRQPFEQFGVAERNGAVGEELRNRSNPINIDNNASSNVDAEAKLLQSFRHCILKLIKLEGSEWLFGQSDGVDEELIDRVAAREKFIYEAEAREINQVGHMGEPLISSVPNCGDGCVWRADLIVSFGVWCIHRVLDLSLMESRPELWGKYTYVLNRLQGVIDPAFSKLRTPMTPCFCLQIPASHQRASPTSANGMLPPAAKPAKGKCTTAVTLLDLIKDVEMAISCRKGRTGTAAGDVAFPKGKENLASVLKRYKRRLSNKPVGMNQDGPGSRKNVTAYGSLG

SEQ ID NO: 2 - protein, 1291 aa, Cucumis melo
MESTTLHTTHQSAAIHRFLPFIAPALLVSISYVDPGKWAATVEGGARFGFDLLVLVLLFNLAAILCQYLSASIGVVTGRGLAQICSEEYDKCTCFFLGIQAEASVILLDLNMILGISHGLNLLLGWDLFTCVLLTGVAAALFPPFADLQEDGRAKFLYICMAGFVLLSLVLGVLISQPEIPLSMNLMPTRLNGESAFTLMSLLGASVMPHNFYVHSSIVQQHQSPPNISKEVLCYNHLFAIFCIFSGIYVVNNVLMNSAANVFYSSGLALHTFPDALSLVEQVFGSSVVYVLFLLVLFLSNQITALTWSLGGQLVLTNFLKLDIPGWLHCATIRIIAIIPALCCVWSSGAEGMYQLLIFSQVMVALLLPSSVIPLYRVASSRTIMGAFKISQLVEFIAIGIFIGILGLKIIFVVEMIFGNSDWVVNLRWNMGSGMSIPFVVLLITACSSFCLMLWLAATPLKSATTIAQLDAEVLNWDMPEVIPDSSEERENIDLGKSSNSAEPIESHSDLSTTKFDFNLPENIMEPDQVLGSVNQNENRSSGVVPSSPKYVQEELESTEELVSSSTVTRDVPDSTLADKKVLKIEPVEPVEKTVGLDGDLRSEKDDYEVDNWEAEESMKEISGNIPSSTSEGPGSFRSIGGKSEEGGNGTGSLSRLAGLGRAARRQLTGILDEFWGQLYDFHGVATQDAKVKKLDLLLGITSLKLDAVGKDFPHSSPLGCKTSDPISSSLYDSPKSQRVQSGLEPPYGIQKGNQPLWSNHMQLWDAYVNNSSHNALDSGVKRYSSLRSLPSTESWDYQPATVHGYQLTYLSRMAKDRSSGNSNGQLDSSGSKYHTLGGGGAGLRDSVAFAMGQKLQNGLGACQQAAPPGFSNIKVSRKPSSESERQYYDLSPSGTGENLVSVSNTKKYHSLPDIHRDQHTSDKSSQWDNATVYGTSIGKITARGVSFANSGSRSVAPLAFDELSPANVYSGALSPQMNPHLDSGSFWHRQPSEQFGLDKNSNSESKGIGRLHSISQEASFVVNSEARLLQSFRDCIVKLLKLEGSDWLFGQSDGTDEELIDCVAAREKFLYEAEAREMGRVVRMKESPSFSPDRRPGSGMKNDTNFSNVSISSVPHCGEGCIWRSDLIVSFGVWCIHRILDLSLMESRPELWGKYTYVLNRLQGIIDPAFSKPRVPMPPCFCLQIPQAFQQRSSPQIANGMLPPAAKPGKGKCTTAAMLLDMVKDVEIAISCRKGRTGTAAGDVAFPKGKENLASVLKRYKRRLSNKPVATHEVSSISRKLSATSVPYSS

SEQ ID NO: 3 - protein, 1275 aa, Dianthus caryophyllus
MAEVLLPAVTPVVLILIGYIDPGKWATYVDVGARYGGDLVVFALLFNVVGVLCHYLSARVTIITGRNLTQICSQEYDRLTCFFLGLQAELSVITLDLTMIIGIAHGLNMIFGLNLFVGILLTALNALLFPFFSSLLESSKAKFVVVCLAGLTIASYVLGALSSLPEFTTSSNLVAKFSGESAFALMGLLGSNVMPHNFYLHSSIVQWYQGQTSVSTSAWSQDNFILNFAISGGIFSATFVLMNSVANGVYSTGVGLLSIQDALSLLDQTYRNSIIPIGAFVVLFLANQIASLSWEFHGEGAKSGEKMMHDFFDMDLPVWIHRAAVRIFAAVIALFCLWHSGAEGMFHLLICTQVIVALLLPSSVIPLFRIASCRPIMDLRKMSPALEFIAILTFMGMLCLELIFVVELIFGESEWVVNLRWTISNGASMSYILLLVAVCVSLFFMFWVAATPLKSSISKLNSQPWNLNAQQVSPGSSIERENNDITETIYSKEESINVEKEVITLEESSLLNHSDTPDANCDINLPDTIMDTVQELYVANSDELPGNSSACHPKPKQLATSSESVAVSSVSTRIEDDTFQKSSNAVNNRMDADEKTLRVEGDSPPEKQDDRNAWEPGESSKGISEVDPSTASDGPGSFRSLSGGGSLSRLSGLGRAARRQMASVLDEFWGQLYDFHGQITQEARSKKLDLLLGADSKPSSQSVSKSNPAGRELVMQSQSLGGRVSGNTINSSLYNSPDQQKLFDSIEAAYKAHRASTSIWSNPPPVSDTYVQNSNRSLLDSGEKRYHSVRLPSSSERSEYQAATVHGYQLASYANRAAKDRSDYAFGRLESVPQKSPSLVPNNYEESFGFTSGRNSENGLHAAQTSSFQNFPVQRRNFDQFDRASYEFSAGPIERMSNHNNAKQYHSSPDISALSARLRNSYLSNGNMQFDSPNTSSGFRATVGRTTYEPSPIRSTGGSTGSRPVGPLAFDELSPSMAYCDAISLSSSSGTRSLWARQPYEQFGLANNTSNLGALAAGNRCTTTAREPPFAEIESKLLQSLRHCILKLLKLEGSEWLFRENDGVDEDLIDRVVTRERFIFEVESREFKQASPLGSSDEAANAHLISSVPHCGEGCVWKLDLIASFGVWCIHRILELSLMESRPELWGKYTYVLNRLQGVIDLAFFKPRTSMSPCFCLQVPASYQRKSTSPFSNDKLPPAIRPAKGKVTTASTILEVIKDVEIAISCRKGRSGTAAGDVAFPKGKENLASVLKRYKRRLSNRAAGANDNGQGLRKL

SEQ ID NO: 4 - protein, 1309 aa, Medicago truncatula
METEALSSEQTKSKMEAETLSTNPPPGFLIRALPAVIPALLISIGYVDPGKWVASIEGGARFGFDLVAFALIFNFAAIFCQYLSARVGVITGRDLAQICSDEYDTWTCLLLGIQAELSVIMLDLNMILGMAQGLNLIFGWDLFTCVFLTATGAVFHILLAILLDIEKTKFLGQFVAGFVLLSFILGVFIQSEVPVSMNGILINLSGESTFMLMSLLGATLVPHNFYLHSSIVQWHQGPANISKDALCHNHFLALLCVFSGLYLVNYILMTTLANEFYSTGPVLLMEQVLHSPIALIGFVLILFLANQTAALTWSLGGEVVVNGFLKLDIPGWLHYATIRVIAVLPALYCVWSSGAEGIYQLLIFTQVLVALQLPSSVIPLFRVALSRSIMGAHKVSQSMELLALTIFLGVLGMNIMFLGEMIFGSSDWACDLRWNLGNGVSVLFSVLLIAGFLSICLMLRLATTPLRSASIQLNAQVLNWDMPEAVLNPPVDGEESHVTETVGHEDASFQADEPKPALARSLEYPEVSLASFRPDLHLPETVMEPDPQVNALKENHSVAPSVSTSDSGTVSKTVANDTSSSDSKLKDTKTIIEANAPIEKTVEIEDDSNVERDDDDVDSWETEESSRAVLANAPSSTSEGPPSFRSISGKSDDGGCSFGSLSRIEGLGRAARRQLAATLDEFWGQLYDFHGQATQAAKAKKIDVLLGMGVDSKPTASLQKMDACGKDYSEYLVSVGGRASDNLINAGPYDYSNQPRMQSNSESAYGLQRSSSSVRASPIQLLDAYVQSSNRNLNDSGERRYSSVRNLHSSEAWDYQPATIHGYQTASYLSRGVKDRSSENINGSMPLTSLKSPSTGNPNYRDSLAFVLGKKLHNGSGVGHPPGFENVAVSRNRQLQTERSNYDSSSPGAAANTVSSVNTKKYHSLPDISGYSIPHRAGYVSDKNAPWDGSVGYGSFAGRMGYEPSMYSNSGSRAGGAHLAFDEVSPYREALSSQFSSGFDTGSLWSRQPFEQFGVAGKIHNVAMEGAGSRPNAIVQEITFEDIEGKLLQSVRLTIMKLLKLEGSDWLFKQNDGIDEDLIDRVAARDKFVYEIEARETNQGIHMGDTRYFPSDRKSVSSMKVNEANASSLSVSSVPNCGEGCVWRADLIISFGVWCIHRILDLSLLESRPELWGKYTYVLNRLQGIIEPAFSKPRTPSAPCFCIQVPTTHQQKSSPPLSNGMLPPTVKPGRGKYTTASSLLELIKDVEIAISSRKGRTGTAAGEVAFPKGKENLASVLKRYKRRLSSNKLVGNQEGTSSRKIPSSGPYNQ

SEQ ID NO: 5 - protein, 1281 aa, Oryza sativa
MDGQQLRSSESPASGGGGVTGGGAPHLFHALGPALLISIGYIDLGKWVAAVEAGSRFGLDLVLLALLFNFMAILCQYLAACIGTVTGRSLAEICHQEYSRPTCIFLGVQAGLSLLTSELTMIFGIALGFNLLFEYDDLITGICFATVVPNLLPYAISHLGKKMVGTLNACIAGFALLCYVLGLLVSQPQIPLTTNVIFPKLSGESAYSLMALLGANVMAHNFYIHSSVVQGQKRSAFAVGALFHDHLFSVLFIFTGIFLVNHVLMNSAAADSTNTLLLTFQDVVELMNQIFVNPMAPTIFLVVLLFSSHIISLTSAIGSQVISQHLFGINLPLSGHHLILKAFAIVPALYCAKVAGAEGIYQLLIICQIIQAMLLPSSVVPLFRVASSRLIMGAHRVSLHLEILTFLAFLLMLFSNIIFMAEMLFGDSGWLNTLKGNTGSPVVFPSTVLITVACVSVAFSLYMAVTPLKSGSHEAELQQEWSVPSQKELLNTTQDREETCAGNVTYEEDQRSDVVPSPRIQPVDCLKSALDYIDSSDTAIESDHDSQHSTAHTSTAPESCHSPSFIPEESKSVVAVDWPEPLEPISNAIVAEESTVESVDSKSTGERDIEVEPALLMDNDKEAPNILESDNKSLGGNNPSCASDDGPPSLTFSRGKGSDAGNGSGSLSRLSGLGRAARRQLAAILDEFWGHLFDYHGKLTQEASSKRFDILLGLDVRTPSSTVRADSQANEIPKSPMVRDNLQGSAFLGSSRDLMSTKNEMSNLDLTYGLQMGNNIGSSAWSQGMQLPSTQLQSSSNSLLDQGARLNSNFSTPSYADNNQFYQPATIHGYQLASYLKQMNANRNPYSSMPLDPQRLPKSSASAVPTYVDSVMHARNQNLLASLGATPSQIAATSRIGTMMAERSYYVPSTLDGNENAGSSAYSKKYHSSPDISALIAASRSALLNESKLGGGTIGSQSYLSRLASERSQYTNSVARPAAPLAFDELSPPKLPGDIFSMQQSPNPSARSLWAKQPFEQLFGVSSAELTKSEFNPAGRSGGMTKDDFSYKESEAKLLQSLRFCISKLLKLEGSGWLFKQNGGSDEDLIDQVAAVEKLLQQGTSDNQLLLGDTQQPPCDKADIQYMRVLPNCGDDCIWRASLVVSFGVWCIRRVLDLSLVESRPELWGKYTYVLNRLQGILDPAFSKPRSALSACACLHRDIRVLNSLRHSSLVATNSIPRQIRGSFTTASVVLEMIKDVETAVSGRKGRSGTAAGDVAFPKGKENLASVLKRYKRRLSSKGQQ

SEQ ID NO: 6 - protein, 1302 aa, Oryza sativa
MDGQQLRSSESPASGGGGVTGGGAPHLFHALGPALLISIGYIDLGKWVAAVEAGSRFGLDLVLLALLFNFMAILCQYLAACIGTVTGRSLAEVADIFVFSTWNKDLPPRIQQANMHLSGCSSRIVLVDIRIDDALGFNLLFEYDDLITGICFATVVPNLLPYAISHLGKKMVGTLNACIAGFALLCYVLGLLVSQPQIPLTTNVIFPKLSGESAYSLMALLGANVMAHNFYIHSSVVQLVSMFANQIISFQGQKRSAFAVGALFHDHLFSVLFIFTGIFLVNHVLMNSAAADSTNTLLLTFQDVVELMNQIFVNPMAPTIFLVVLLFSSHIISLTSAIGSQVISQHLFGINLPLSGHHLILKAFAIVPALYCAKVAGAEGIYQLLIICQIIQAMLLPSSVVPLFRVASSRLIMGAHRVSLHLEILTFLAFLLMLFSNIIFMAEMLFGDSGWLNTLKGNTGSPVVFPSTVLITVACVSVAFSLYMAVTPLKSGSHEAELQQEWSVPSQKELLNTTQDREETCAGNVTYEEDQRSDVVPSPRIQPVDCLKSALDYIDSSDTAIESDHDSQHSTAHTSTAPESCHSPSFIPEESKSVVAVDWPEPLEPISNAIVAEESTVESVDSKSTGERDIEVEPALLMDNDKEAPNILESDNKPLGGNNPSCASDDGPPSLTFSRGKGSDAGNGSGSLSRLSGLGRAARRQLAAILDEFWGHLFDYHGKLTQEASSKRFDILLGLDVRTPSSTVRADSQANEIPKSPMVRDNLQGSAFLGSSRDLMSTKNEMSNLDLTYGLQMGNNIGSSAWSQGMQLPSTQLQSSSNSLLDQGARLNSNFSTPSYADNNQFYQPATIHGYQLASYLKQMNANRNPYSSMPLDPQRLPKSSASAVPTYVDSVMHARNQNLLASLGATPSQIAATSRIGTMMAERSYYVPSTLDGNENAGSSAYSKKYHSSPDISALIAASRSALLNESKLGGGTIGSQSYLSRLASERSQYTNSVARPAAPLAFDELSPPKLPGDIFSMQQSPNPSARSLWAKQPFEQLFGVSSAELTKSEFNPAGRSGGMTKDDFSYKESEAKLLQSLRFCISKLLKLEGSGWLFKQNGGSDEDLIDQVAAVEKLLQQGTSDNQLLLGDTQQPPCDKADIQYMRVLPNCGDDCIWRASLVVSFGVWCIRRVLDLSLVESRPELWGKYTYVLNRLQGILDPAFSKPRSALSACACLHRDIRVLNSLRHSSLVATNSIPRQIRGSFTTASVVLEMIKDVETAVSGRKGRSGTAAGDVAFPKGKENLASVLKRYKRRLSSKGQQ

SEQ ID NO: 7 - protein, 1188 aa, Oryza sativa
MHLSGCSSRIVLVDIRIDDALGFNLLFEYDDLITGICFATVVPNLLPYAISHLGKKMVGTLNACIAGFALLCYVLGLLVSQPQIPLTTNVIFPKLSGESAYSLMALLGANVMAHNFYIHSSVVQLVSMFANQIISFQGQKRSAFAVGALFHDHLFSVLFIFTGIFLVNHVLMNSAAADSTNTLLLTFQDVVELMNQIFVNPMAPTIFLVVLLFSSHIISLTSAIGSQVISQHLFGINLPLSGHHLILKAFAIVPALYCAKVAGAEGIYQLLIICQIIQAMLLPSSVVPLFRVASSRLIMGAHRVSLHLEILTFLAFLLMLFSNIIFMAEMLFGDSGWLNTLKGNTGSPVVFPSTVLITVACVSVAFSLYMAVTPLKSGSHEAELQQEWSVPSQKELLNTTQDREETCAGNVTYEEDQRSDVVPSPRIQPVDCLKSALDYIDSSDTAIESDHDSQHSTAHTSTAPESCHSPSFIPEESKSVVAVDWPEPLEPISNAIVAEESTVESVDSKSTGERDIEVEPALLMDNDKEAPNILESDNKPLGGNNPSCASDDGPPSLTFSRGKGSDAGNGSGSLSRLSGLGRAARRQLAAILDEFWGHLFDYHGKLTQEASSKRFDILLGLDVRTPSSTVRADSQANEIPKSPMVRDNLQGSAFLGSSRDLMSTKNEMSNLDLTYGLQMGNNIGSSAWSQGMQLPSTQLQSSSNSLLDQGARLNSNFSTPSYADNNQFYQPATIHGYQLASYLKQMNANRNPYSSMPLDPQRLPKSSASAVPTYVDSVMHARNQNLLASLGATPSQIAATSRIGTMMAERSYYVPSTLDGNENAGSSAYSKKYHSSPDISALIAASRSALLNESKLGGGTIGSQSYLSRLASERSQYTNSVARPAAPLAFDELSPPKLPGDIFSMQQSPNPSARSLWAKQPFEQLFGVSSAELTKSEFNPAGRSGGMTKDDFSYKESEAKLLQSLRFCISKLLKLEGSGWLFKQNGGSDEDLIDQVAAVEKLLQQGTSDNQLLLGDTQQPPCDKADIQYMRVLPNCGDDCIWRASLVVSFGVWCIRRVLDLSLVESRPELWGKYTYVLNRLQGILDPAFSKPRSALSACACLHRDIRVLNSLRHSSLVATNSIPRQIRGSFTTASVVLEMIKDVETAVSGRKGRSGTAAGDVAFPKGKENLASVLKRYKRRLSSKGQQ

SEQ ID NO: 8 - protein, 1304 aa, Prunus persica
MANLESANPSANNMLGVLHRLLPVVGPALLISVGHLDPGKWAATAEAGARFGSDLAALMLIFNFAAILCHYLSARIGVVTGRDLAQICSEEYDKGTCIFLGVQTEVSVILSDLTMILGIAHGLNLLFGWDLFTCVFLTAVNAVLYPLFSTLLETCKAKVLCVCIAGFIQLSFVLGVIISQPEMSFSMNGMLTKLSGESAFALMSLLGASIMPHSLYLHSSIVQQYQCQPTVSRDALCHHHLVAILCIFSGIYLVNYALMTSAENEYSGLGLLTFQDVMSLIGQVFWGPIVSGAYLLVLFVSNQITTLSWSLGGQVVLNDFLKLDLPGWLHCATIRIIAIVPALYFVWSSGAEGMYQLLIFTQVLAALLLPSSVIPLFRIAASRPIMGVHKVSQFVEFLSLITLIGMLGLKIIFVVEVIVGNSDWVNNLRSNAGSSMSVPCVLLLTACATFCLMIWLAATPLKSASARLEAQVWIWDMHMGSPDSITKKEEINISEPKYHREVSVQKHEPSPSFGRALDSDSEVASFDLDLPETITEPDEEHHLTTVAENGSRITFPHSPKCHMEGSTSTVESTPVSTVVNEVSDVTLEGTSALKIESTEPIEKTVGVEGVEGDLPNEKDDDEGDTWEPEDSLKGVSESTAPLTSEGPGSFRSLSGKGDEGGSSAGSLSRLAGLGRAARRQLAAVLDEFWGQLYDFHGNVIQEAKAKKLDLLLGLDSKAASSSLKVDTSAKELSGYFPSAGGRGSDPIMNSSLYDSPKQQRVQSSLESYGVQRGSSALLPSRVQLLDAYVQNSSRSVIDSGERRYSSVRSLPSSESWDYQPATIHSYHPSYLNRIAKDRGFDNLNGQMESAALQSASSLGAANYRDSLAFTMGQKLQNGLGSGQASIFQNHTVSRNSPLQSERPYYDLHPSGIAENVVSSANAKKYHSLPDIHRDLYMPEKSANWESPVGYGSSTGITNYESSLYSNSGARTGAPLAFDQLSPSQVYRDAFSSQQNSSFNTGSLWSRQPFEQFGVADNNRTIGSGGFGYRAGSVSQEATSVADSEAKLLQSFRHCIVKLLKLEGSDWLFTQNDGVDEDLIDRVAAREKFLYEAETREMNRTVHMGEPQYHPSDRKSVSALKNNDANCTSFMVPTCGEGCIWRSDLIVSFGVWCIHRILDLSLMESRPELWGKYTYVLNRLQGIIDSAFSKPRTPMSPCFCLQISAVHQLKSSPSFSNGIPPAAKPARGKCTTAVTLLDIIKDVEIAISCRKGRTGTAAGDVAFPKGKENLASVLKRYKRRLTNKTAGAHEGPGSRKVQTSAPYGS

SEQ ID NO: 9 - protein, 1220 aa, Ricinus communis
MESEFVNANHLPGTIHRLLPSVGPVILVALGYVDPGKWAATVEGGARFGHDLIVPMLIFSFAAILCQYLSARIGVVTGRDLAQICSAEYDKFTCMFLGVQTALSVIALDLTMIIGIAHGLNLLFGVDLSTGVFLTAVDAVNFYLHSSFVLQQPGGRIVSKDTLCLHHFFAILCVFSGIYLLNYVLMNSAANVFNSTGLVLLTFPDAMSLMEQVFRNPMAPLAFLIILYFTNQLTALTWNLGGQVVLHDFLRLDIPNWLQHATIRIMAIVPALCCVWTSGVEGIYQLLIFTQVMTALLLPSSVIPLFRVASSRPIMGVYKISQILEFLALVTFMGLLGLKIIFVVEMIFGDSDWVSNLRWNMGSSASIPYVALLITACSSFCLMLWLAATPLKSATLLDAQAWTCDISNVPETSTQRKENFVSEILHNGGEPIQNQEQLPALENSLENYSDIAGPNTELDLPETIMESDNELHLTTAEENYCDVKFHNPPKSYQEESTSIMDKVPVSTIVNEVADGDLPDTEKIQIESMEPIEKTVGIEGESQAEKEDDEGETWEPEEPSKAAPGSLSSLAPDGPPSFRSLSGKSDEGGNGAGSLSRLAGLGRAARRQLAAVLDEFWGQLYDFHGQVTQEAKNKKLDLLLGESKLASSSLNVDITGKDFSGYFPSSVGRGSDSLMNTSLCDSPKQLRVQSNVDSSYGVQRGSSSMWSNHMQLLDAYVQGSSRNVVDATERRYPSVRTLPSSDGWDNQPATVHGYQIASIVNRLAKDRNPNDLNGQMESPAPISPSLGPRNYRDPLAVALGQKLQNGLSSPQASRYQNFPTSGNSSLQSERPYYAVCSSGSADSTGMSANTKKYHSLPDISGISGPYRDLYMSEKSNQWDNTVGFGASVGRTSYEPSFYSNTGMGAGGALAFDNVSKGYRDAFSYSVSSERGSIWSKQPYEQFGIANKSRTVGSGLGSRSNSITREAISVADSEAQLLQSFRCCIVKLLKLEGSDWLFRQNDGADEDLIDRVAARERCLYEVETREINRMVQIGEPQYSYSDTKSGSALKNDETGIANIPVSSVPHCGEGCVWKADLIISFGVWCIHRILDLSLMESRPELWGKYTYVLNRLQGIIEPAFSKPRGPMSPCFCLQLSAAYQRKSSPPVTNGMLPPAAKPGRGKCTTGAMVLDLIKDVEIAISCRKGRSGTAAGDVAFPKGKENLASVLKRYKRRLSSKPIGSK

SEQ ID NO: 10 - protein, 1316 aa, Solanum lycopersicum
MESETLTREYRRPSMLQRVLSASVPMLLIAVGYVDPGKWAAMVDGGARFGFDLVMLVLLFNFAAILCQYLSACIALVTDRDLAQICSEEYDKVTCIFLGIQAEVSMIALDLTMVLGTAHGLNVVFGVDLFSCVFLTATGAILFPLLASLFDNGSAKFLCIGWASSVLLSYVFGVVITLPETPFSIGGVLNKFSGESAFALMSPLGASIMPHNFYLHSSIVQQGKESTELSRGALCQDHFFAIVFIFSGIFLVNYAAMNSAANVSYSTGLLLLTFQDTLSLLDQVFRSSVAPFTIMLVTFISNQVTPLTWDLGRQAVVHDLFGMDIPGWLHHVTIRVISIVPALYCVWSSGAEGLYQLLILTQVVVALVLPSSVIPLFRVASSRSIMGIHKISQLMEFLSLGTFIGLLGLKIIFVIEMIFGNSDWVNNLKWNIGSSVSTPYFFLLIAASLCLCLMLWLAVTPLKSASSRFDAQAFLQTHVPEPYSECNQLGASNAMFGLVEGSSQKQEGAFHVEKSLVSHPDLSTKDPDQLLPESLLDFEKVHQLATIDESKSETTFSAPAVVHPEVPVSAGASPSVKSVCNEVSGVVSVDTSVFNTETVDVAEKTLRIEGDMANDRDDGDSWEEPEEAIKGVSENAQSFISDGPGSYKSLSGKLEDTGSGTGSLSRLAGLGRAARRQLTEALNEFWGQLFDYHGVATAEAKSKKLDIILGLDSKMNPKPAPASLKVESSAYIPSGSARIPEPLINSHVYSPKQQFASNIVDSAYRVPKEPSSTSSMWSNHMKLVGAYVQSSNSNMLDSGERRYSSMRIPATSAGYDQQPATVHGYQITAYLNQLAKERGSDYLNGQLESPSPRSVSSLTSNYAEPLARVSGQKPQSGVSSRAPPGFGNVPVGRNNSMQPTNTTSVDHSSTETAESVAGSANSKKYYSLPDISGRYVPRQDSIVSDARAQWYNSMGFGQSGGRSTYEQAYMSGSLRAGGPQRYEHSPKVCRDAFSLQYSSNSGTGSLWSRQPFEQFGVAGKPDVGSGDHGTVLSSSAQESTSTVDLEAKLLQSFRSCIVKLLKLEGSEWLFRQDDGADEDLIGRIAAREKFLYEAETREISRLTNIGESHFSSNRKPGSAPKPEEMDYTKFLVMSVPHCGEGCVWKVDLIISFGVWCIHRILELSLMESRPELWGKYTYVLNRLQGIVDLAFSKPHSPTSHCFCLQIPAGRQQKASPPPISNGNLPPQAKQGRGKCTTAAMLLEMIKDVETAISCRKGRTGTAAGDVAFPKGKENLASVLKRYKRRLSNKPVGNQEVAGVAGPRKVTLSASSPPFVL

SEQ ID NO: 11 - protein, 1316 aa, Solanum lycopersicum
MESETLTREYRRPSMLQRVLSASVPMLLIAVGYVDPGKWAAMVDGGARFGFDLVMLVLLFNFAAILCQYLSACIALVTDRDLAQICSEEYDKVTCIFLGIQAEVSMIALDLTMVLGTAHGLNVVFGVDLFSCVFLTATGAILFPLLASLLDNGSAKFLCIGWASSVLLSYVFGVVITLPETPFSIGGVLNKFSGESAFALMSPLGASIMPHNFYLHSSIVQQGKESTELSRGALCQDHFFAIVFIFSGIFLVNYAAMNSAANVSYSTGLLLLTFQDTLSLLDQVFRSSVAPFTIMLVTFISNQVTPLTWDLGRQAVVHDLFGMDIPGWLHHVTIRVISIVPALYCVWSSGAEGLYQLLILTQVVVALVLPSSVIPLFRVASSRSIMGIHKISQLMEFLSLGTFIGLLGLKIIFVIEMIFGNSDWVNNLKWNIGSSVSTPYFFLLIAASLCLCLMLWLAVTPLKSASSRFDAQAFLQTHVPEPYSECNQLGASNAMFGLVEGSSQKQEGAFHVEKSLVSHPDLSTKDPDQLLPESLLDFEKVHQLATIDESKSETTFSAPAVVHPEVPVSAGASPSVKSVCNEVSGVVSVDTSVFNTETVDVAEKTLRIEGDMANDRDDGDSWEEPEEAIKGVSENAQSFISDGPGSYKSLSGKLEDTGSGTGSLSRLAGLGRAARRQLTEALNEFWGQLFDYHGVATAEAKSKKLDIILGLDSKMNPKPAPASLKVESSAYIPSGSARIPEPLINSHVYSPKQQFASNIVDSAYRVPKEPSSTSSMWSNHMKLVGAYVQSSNSNMLDSGERRYSSMRIPATSAGYDQQPATVHGYQITAYLNQLAKERGSDYLNGQLESPSPRSVSSLTSNYAEPLARVSGQKPQSGVSSRAPPGFGNVPVGRNNSMQPTNTTSVDHSSTETAESVAGSANSKKYYSLPDISGRYVPRQDSIVSDARAQWYNSMGFGQSGGRSTYEQAYMSGSLRAGGPQRYEHSPKVCRDAFSLQYSSNSGTGSLWSRQPFEQFGVAGKPDVGSGDHGTVLSSSAQESTSTVDLEAKLLQSFRSCIVKLLKLEGSEWLFRQDDGADEDLIGRIAAREKFLYEAETREISRLTNIGESHFSSNRKPGSAPKPEEMDYTKFLVMSVPHCGEGCVWKVDLIISFGVWCIHRILELSLMESRPELWGKYTYVLNRLQGIVDLAFSKPHSPTSHCFCLQIPAGRQQKASPPPISNGNLPPQAKQGRGKCTTAAMLLEMIKDVETAISCRKGRTGTAAGDVAFPKGKENLASVLKRYKRRLSNKPVGNQEVAGVAGPRKVTLSASSPPFVL

SEQ ID NO: 12 - protein, 1272 aa, Solanum lycopersicum
MDATDVQQSMGFMESRGGMPKFFHALGPALLISMGYIDLGKWVAAVEAGSRFGFDLVLLALLFNFTAIVCQYLAACIGTVTGKNLAEICHQEYNQPTCIFLGVQAGLSLLTSELTMIFGIALGFNLLFEYDDLITGICFATVVPNLLPYAISHLGKKMEGTVNACIAGFALLSYVLGLLVSQPQIPLTMNVIFPKISGESAYSLMALLGANIMAHNFYIHSSVVQGQKKSSAVGLGALFHDHLFSILFIFTGIFMVNYVLMNSAAAESTNTLLITFQDVVELMNQIFVNPLAPTIFLVVLLFSSHIISLTSAIGSQVISQHLFGINLPLSGHRLLLKVFAIVPTLYWAKVAGAEGIYQLLIICQIIQAMLLPSSVIPLFRVASSRSIMGAHRVSLHLEILVFLAFLLMLFSNIIFVAEMLFGDSGWMNNLKGYTGSPVVLPYTVFILVACVSVAFSLYLAVTPLRSGSHEAESHEWSVHSQRELLNTPQEREDVKVDNVTYEEDQRSDVGPSPRDAPDSHPELAMDYIDTSDTAVESDHDSQQSTAYASTAPETCPSPSFTREESKSVVAVNWPEPLEKVPTSTVIEESTVESVVSRITTERDVLVETDVFSGKDKEDTHVLESEKSIVDSTPCVSDDGPPSLTFSRGKGSDAGNGNGSLSRLSGLGRAARRQLAATLDEFWGHLFDYHGKLTQEASTKKFGILLGIDLRTPTTAVRTDKQAVEIPKSPLVRDSMRGAAFLSSSVDLMSPKNETSNLELAYGLQRGPAMGLSSWSQGMQLPNTQLQSSSNSLLEQSARLNSNFSAPSYSDNNQFYQPATIHGYQLTSYLKQMNASRNPYSSMPLDPQRLPKSSVSAVPTYVDSMMNARNHNLLASLGATPSQIPATSRVGSMMPERSYYDPSTVDGNENSGSPAYSKKYHSSPDMSGIIAASRAALLNEAKLGGAIGPQSYLSRLASERSQYANSAARPAAPLAFDELSPPKLQSDIFSAQSSMSPSARSLWAKQPFEQLFGMSSAELSKGDFNLSGRSGGMAKDDFSYKESETKLLQSLRFCIMKLLKLEGSGWLFKQNGGCDEELIDRVAASEKLLMQGTTENQLLHGDLQQHTSDQVGIQYMRTLPNCGEDCVWRASLVVSFGVWCIRRVLDMSLVESRPELWGKYTYVLNRLQGILDPAFSKPRSALTICACLQKDIRVLNSPPGSGLTAMGPIPIPIRGTFTTAGVVLETIKDVETAVSGRKGRSGTAAGDVAFPKGKENLASVLKRYKRRLASKGQ

SEQ ID NO: 13 - protein, 1258 aa, Zea mays
MDAPDVQQSMGYKESRGGMPKFFHALGPALLISMGYIDLGKWVAAVEAGSCFGFDLVLLALLFNFTAIVCQYLAACIGTVTGKNLAEICHQEYNQPTCIFLGVQAGLSLLTSELTMIFGIALGFNLLFEYDDLITGICFATVMEGTINACIAGFALLSYVLGLLVSQPQIPLTMNVIFPKISGESAYSLMALLGANIMAHNFYIHSSVVQGQKKSSAVGLGALFHDHLFSILFIFTGIFMVNYVLMNSAAAESTNTLLITFQDVVELMNQIFVNPLAPTIFLVVLLFSSHIISLTSAIGSQVISHHLFGINLPLSGHRLLLKVFAIVPTLYWAKVAGAEGIYQLLIICQIIQAMLLPSSVVPLFRVASSRSIMGAHRVSLHLEILVFLAFLLMLFSNIIFVAEMLFGDSGWMNNLKGYTGSPVVLPYTVLVLVALISVAFSLYLAVTPLRSGSHEAESHEWSVHSQRELLNTSQEREDVKVDNVTYEEDQRSDVVPSPRDVPDSHPELALDYIDTSDTAVESDHDSQQSTAYASTAPETCSSPSFTREESKSVVAVNWPEPLEKVPTSTVMEESTVENVVSRITTERDVLVETDVVSGKDKEDIRTLESEKSIVDSTPYVSDDGPPSLTFSRGKGSDAGNGSGSLSRLSGLGRAARRQLAATLDEFWGHLFDYHGKLTQEASTKKFGILLGIDLRTPSTSVRTDKQAAEILKSPLVRDSMRGAAFLSSSVDMMSPKNETSNLELAYGLQRGPGMGLSSWSQGMQLPNTQLQSSSNSLLEQSARLNSNFSSSYSDNNQFYQPATIHGYQLTSYLKQMNASPSLYSSMPLDPQRLPKSSVSAVPNYADSMMHARNHNLLASLGGTTTQLPATSRVGSMMPERSYYDPSSVDGNENAGSPAYSKKYHSSPDMSGIIAASRAALLNEAKLGAAIGPQSYLSRLAAERSQYASSTARPAAPLAFDELSPPKLQSDIFSAQSSMRPSARSLWAKQPFEQLFGMSSAELSKGDFNLPGRSGGVAKDDFSYKESETKLLQSLRLCIMKLLKLEGSGWLFKQNGGCDEDLIDRVAAAEKLLMQGTAENQLLLHGGDLQQHSSDQAGIQYMRTLPNCGEDCVWRASLVVSFGVWCVRRVLDMSLVESRPELWGKYTYVLNRLQGILDPAFSKPRGALTICTCLQKDTRVRNSPPHSGLTAMGPVPTPIRGAFTTAGVVLEMIKDVEAAVSGRKGRSGTAAGDVAFPKGKENLASVLKRYKRRLASKGQ

SEQ ID NO: 14 - protein, 1251 aa, Selaginella moellendorffii
MRIGVPSGSYISLLAPALLVSIGSIDPGNWATSIEGGSRFGYELVWVVFLANLIALLLRSLATHLNLVSGKHLAQACHDEYPSGVCLLLLFLSELSLVVLDSAMVLSSAVGLNMVLGLPVLVGALLVALDVFFLMGFVPFLGPGKAEVILGVIVLSMVLVFVLNAFVSRPPVVPILGGLWPRLKSDSLYAVVGLLGANIMPHNLYLNSALGQEQRKPDRICCAPTILDFILTAGMPAAINLAVMVVAASTFHSAGFAVLTLQDGCVVMEQALSRSVAPLAFGSALLAAGVFSTLTGTLVSQVASEGLLGKRIDAWRHRLVMRAAAVTIAVFCAWSYGNEGIYQLLVLSQVILALQLPFTLVPLIRLTSSAAYMGKNKISSLWEVLAWSVLAFVISLNLCLVFSTLFSQSEEFGGSTNWEFVAGSDSNSLTVPVALTVVTAAVGFLVWLIFAPIRSDTPAFAVEEKDGQLMDKEKDLVYYIPLDGNSSRSSDVGTVEDIINTERAELISESEVGTVTVVESEADECLPVLQQSKVDASHLSDSVREDTGEEEASITATSNSSDVGTEECPSSRVDHHHIDHHIEPEPTSVPTNDNVPYSSGETSAVASSEIGGLDKDAISLEKDDEEAESWENLEQDCAVMSSALSLTYEGIESVISTRKESSDGCSHGSESGSLSRLSGLGRASRRQFAATLDEFWGRLFDLHGQPVRSDSVIKPSVSQNSRTTHSDNGPFVSPRQAAKRDYKSCFSGSVDPFGRVHSNLAASTSSQSPSNRWCFDYKSGYSGGDQRYSSLRSFAAHEDLDCQPATIHGYNIASYTRPGSVEQQSSARARSSLHLDVASRTGGCLSQVPASYYAKSNFDQSDTMTRVFQPTQDMFSASKGGQRGVDDTSSWLPRSTDYEQWSSFSNNSVDKWNPLVYRATGELDKSLLSHPIGRSENSERTTLSFDDISPSQTHRDGFSIQAAAQQTESLWSRPVEHLFGAPGEQKSVATTNNANNNTKAFRLSTGVEFGLDTLEQLRLCVRKLLQQEGSEWLFQIDNGCDEDVIAGVAAREKSLLDLSEPGEAASVYSNGTSIWSVNKVVVPGVPHCGDSCVWGSGLLVSFGVWCVHRVLELALMESRPELWGKYTYVLNRLQGILDPAFLSPRTVLPPCICLQGYQLVGRRNDAAGWSSYPFPWPWGRNTSNPSGKAASGNLYLDMIKEVETAVGARKGRTGTAAGDVAFPKGKENLASVLKRYKRRLGNHKSGSCNRR

SEQ ID NO: 15 - protein, 1280 aa, Physcomitrella patens
MAKYVPALAPIVLIAIGYMDPGNWASAIEGGSRFGFELVWVVILSNVMAALFQTLATRLGLVSGKHLAEVLIFLPQLWLPRAVCCIVLGIKSGVVILCFGLGTLLSRPSAFAFLGGVWPKVRGDSLYTAVGLIGANVVPHTFYLHSALLQGQHKLGSKAKELVVQENMRDTIGAFAIAMLANLAVLVVAASTFHNVGLVILTLQDAYALMEQVFYFNMLSLVQILSNSIAPAVFGLALICAGQLSSLTGTLTGKVTMEGFLDMSMGPWLHRSLVRGAAVIPAAFCAWRYGSEGLYRLLLFAQIIIAMELPFSALPLIKASASEARMGSYKISSLVESVAWISVALVVAANIWMVFDVLLDEIDEFAGFAAHLESLLGTDSFGHYGEDTLNTSVFALVLIGIGLCVGFLIWLVVTPLRIDRIAMERKWVQEYELFERNEVMRGSQDIVEDSLRVVEYPPDPAITEVYDMIPITQDVMLANDLLDLEYSSRPESALSGLDGDSALELGRGEVGRGELAFWPSTVSTSSSDIDDLVSKSTELAYPPEPVSQGARSDVSEGSVGGSGSGSLSRLSGLGRAARRQFAAYLDEFWGKLFDLHGQPIASKVGTRTNAIGSTAGPQSSPYDNPCIQTRSVDPGAYLPRVSSPADVCLGGENNGYSLRNQREPLKSTNSLHLPVDQHLEDAFARAQSHTTRTTSSGFPGLPDFSVRSGSLGSPYLPERQYSSMRLPSFSEEIDRQPATIHGYHVPSFLGGRSATSSPANKSTRPMSSFEVYGSESVRTLSGQASVQQPLGIGVQRDFEGSLYGGGFHGSSEDARQSIRSMNALPNRSAMNHRVTGERDLNGPKSWDSRMTTLDPLVYRATGELYQPSSGHSPIGRLGSTSRGGAPLSFDDLSPSVSHRDGFSIQSAQSENSLWSRQPFDQLFGGVTERTTDGESGRSRGGSGSMGSSRSGNINGRSGKTGSGSDSLSNASTAFSSWPDTDLEMMDNLRTCICKLLCLEGSEWLFRFDNGSDEDLVAAVAAVEKMHLEADAPDRVSRNERWQAHGRPVSSLLKSSNEARRVSYCGESCVWGKGLLVSFGVWCVHRVLELSLMESRPELWGKYTYVLNRLQVPPCFQQRIISCFCVQGVLEPAFSKLRLVAPMCNCVLEVSEILNMRAVRGGLTKQGSNSFRVDSLNGDHLNSPQSSYPYPSSWGRNSSSTKGKGASATVFLEMIKEVEQAVGSRKGRTGTAAGDVAFPKGKENLASVLKRYKRRLGNKPPGAAGGGGGGGGGGGGGGTGG

SEQ ID NO: 16 - protein, 1285 aa, Physcomitrella patens
MAKHVPALAPIILVAIGYMDPGNWACAIEGGSRFGFELLWVVILSNCMAAFFQTLATRLGLVSGKHLAEVAEMVAGTIVGVVLVCFGLGALLSGPTAPAIISGWPKVRGDSLYIAVGLIGANVMPHTFYLHSALLQGQHKLSSKAKEIVVQENMRDTIGAFGVAMLANVAVLMVAASVFHNVGLVILTLQDAHALMEQVFILSNSVAPAVFGLALLCAGQLSSLSGTLTGKVTMEGFLDMSMRPWLHRSLIRGAAVIPAAFCAWHYGSEGLYRLLLFAQIVIAVELPFSAVPLVKASASESRMGSYKIPSLVESIAWVSVALVVVANIWMVFDVLLDEIDEFSGFAAHLESLLGTDSFGHHGEDTLNTSVFALVLIVIGLSVGFLIWLVVTPLRVDRIAMERKWVQEYELFESQTLSDDRLGVLEYSSDPTTSEVYDMIPMTEDIMLVNNLVGLEYPSRQESALSSIDTDPALELGRGELVKWTSTVSTSSSDIDDIVSNQPELVYLSEPAQLAADEAESIALKRAEAEADADLIDKDDDEVDDFDHEDVVVGPLSHDNLRRSYSNLAYEASGSARSLGGRTDASEGGGSGSGSLSRLSGLGRAARRQFAAYLDEFWGKLFDLHGQPIASKGSTRLSALGSSIGPLSLPFDNPGITPRSGDPGAYLGRLNSPPDGYLGGESNGYGSKRGPSKSTSSLHQQVEQHFMDAYARAQSHATATTSSGFLGFMHDYSGRSGNSGSLQLPERQYSSMRLPTFSDEFDREPATIHGYYAPSFLGGRSATSSPALKSTRPMSSLDMHGAASARALSGQLHVQQPLGVGVQGEFEGSLYGGGFQGSLEDAIQSLRSMHALPSRNAMNCKVNADRELNGPRSWDSRMATVDPLVHRATGEIYQPSSGHSPIGRIGSMSSVRAPLSFDELSPSVTHRDGFSIQTAQSENSLWSRQPFEQSFSGMTDPSANGGSSRSRGGSGGLGTNRNGTVGGRPGKTSGGAESLSNASTAFSSGLDADLDMMETLRACICKLLCLEGSEWLFRFDNGSDEDLVAAVAAVEKMHMDADSPDRVSRNERWQAHGRPVSSFLKGSNDARRVCYCGEACVWGKGLLISFGVWCVHRVLELSLMESRPELWGKYTYVLNRLQVLSHPQGVLDPAFSKPRLVAPMCNCVIEVSEILSRRAPRGGLIKQGSNGFRIDSTNGDHVSLPQSSYPFPWSWGRNSSSSKGRGASATVFLEMIREVEQAVGSRKGRTGTAAGDVAFPKGKENLASVLKRYKRRLSNKPPGAAGGGGG

SEQ ID NO: 17 - protein, 1310 aa, Petunia X hybrida
MESETQTIAYRQPSMLQRILSASMPMLLIAIGYVDPGKWAAMVDGGARFGFDLIMLALLFNFAAILCQYLSACIALVTDQDLAQICSEEYGKVTCIFLGIQAEVSMIALDLTMVLGTAHGLNVVFGVDLFSCVFLAATGAILFPLLASLLDNGSAKFICIGWASSILLSYVFGVVISQPESPFSIGGMLNKFSGESAFALMSLLGASIMPHNFYLHSSIVQQGKESTNLSRGALCQDHFFAIVFVFSGIFLVNYAIMNSAANVSFSTGLLLLTFQDSLSLLDQVFRSSVAPFSIMLVTFISNQITPLTWDLGRQAVVHDLFGMDIPGWLHHVTIRVISVVPALYCVWNSGAEGLYQLLIVTQVVVALVLPSSVIPLFRVASSRSIMGIHKISQLMEFLSLGTFIGLLGLKIIFVIEMIFGNSDWVNNLKWSIGSGVSTPYVFLLIAASLSLCLMLWLAVTPLKSASSRFDAQAFLQTPMPESYREHNQVDVSDTTFGLERSTQKQEPAFHVEKSLGSHPDLSTSDPDEILPESLLDFEKVHHLTTIDESKSETTFSTPSFSCPEVSASAGETAKSVLNEVSGGESVDTRDFNAASVDVVEKTLRIEGDTPTDKDDDGDSWEPDDVPKDVSENTQSYTSDGPESFKSLSVRSEDTGSGTGSLSRLAGLGRAARRQLTVVLDEFWGQLFDYHGMPTSQAKFKKLDVILGLDTKVDPKPAPVSLKLENSRGDSNAYIPSGSARVPESWINSNIYSPKQQCASGALDSGYRVPKEPASWSSHMKLLDAYVQSSSGNTLDSGERRYSSMRIPASSAGYDQQPATVHGYQISAYLSQIAKGRGSDYLNGQLESASPRSVSSLTSNHAEPLARALGQKPQSGVSSRAPPGFGSVPARNNSMQPVNTSTDLSSTENAESVAGSANSKKYYSLPDISGRYVPRQDSSLPDGRAQWYNSMGYGQSIGRSAYEQPYMTGPMRAGGPPRFEHSPSKVCRDAFTLQYSSNSGTGSLWSRQPFEQFGVAGKADVSSDHGTVQSSSTQESTSLVDLEAKLLQSFRSCIVKLLKLEGSEWLFRQDDGADEDLIDRIAAREKFLYEAETREISRLTNIGESQFSSNRKPGSAQKPEEMDYTKFLVMSVPHCGEGCVWKVDLVVSFGVWCIHRILELSLMESRPELWGKYTYCLNRLQGIVDLAFSKPRSPTSHCFCLQIPIGRQQKSSPTPISNGSLPPQAKQGRGKCTTAPMLLDMIKDVEMAISCRKGRTGTAAGDVAFPKGKENLASVLKRYKRRLSNKPVGNQEAGGGPQRKVTSPSSTSFGL

SEQ ID NO: 18 - protein, 1259 aa, Populus trichocarpa
MDTEFANANHPLHFLHRLLPAVGPGLLIAIGYVDPGKWAATVEGGARFGFDLVLPMLLFNFVAILCQYLSARIGVITRKDLAQICNDEYDKWTCMFLGVQAALSVIALDLTMILGIAHGLNLLFGMDLSTCVSLAAAEAILFPFFATLMERCKASFLCTCIAGFILLLYFFGVLISQPGIPLSINGTRTKLSEESVFALMSLLGASIMPHNFFLHSAIVLQHQGPPNISRDALCLNHFFAILCIFSGIYLVNFVLMNSAANVFHSTGLVLLTFPDAMSLMEQVFRSPVAPFGFSLILFFANQITAFSWNLGGQVVLHNFLRLDIPNWLQRATFRIIAVVPALYCVWTSGVEGIYQLLILTQVMVALLLPSSVIPLFHIASSRQVMGVYKISPFLEFVALISFMGMLGIKIIFVVEMVFGDSDWVGTLRWSTVSGSSTSYIVLLITACSSFCLMLWLAATPLKSATRLDAQVCNWDVQNAVSEPSTLIEEEFLTENICTGEELIERQEQLPEPGKSFESYSNITVANADPDLPETIMESDQELHLTTIKEKHSEVAFSSPQTFYEETSPTTESASLSASVNLVPDAELLVAKKAKIESMDPVEKTLDIEGELHTEKEDDEGDNWEPEDSSKGVPGSTLSLTSDGPGSFRSLSGKSDAGGNGAGSLSRLAGLGRAARRQLAAVLDEFWGQLYDFHGQITQEAKTKKLDALGVDLKLASSQLKVDTAGKESSGYFSLVGGRASDSLINSSLCDSPKQLRVQSNIDSSYGVQRGPSSLWSNHMQLLDAYVQGPSQSIADSSERRYSGVRTPPSSDGWDNQPATVHGYQIASIANRIAKDRGFSSLNGQMESPAPISPSLGPRNYRDPLTVSMGKNLQNGLSSSQASGFQNLAVTRNSPLQSERPYHDVYSGSADDTGMSANTKKYHSLPDISGLAGPYRDLYMSEKNAQWDKSAGFGSSVGRSAYEQSYYSNTGSGAGGPLSFNGLSKGHGDAFSLHMTPDPGSLWSKQPFEQFGVADKIRAVGSGLGNRSNSINREVTSPVDSEAQLLRSFRHCIVKLLKLEGSDWLFRQNDGADEDLIDCVAARERYLYEAETREMNHVDHMVPHCGEGCVWRSDLIISFGVWCIHRILDLSLMESRPELWGKYTYVLNRLQGIIELAFSKPRTPMSPCFCLQIPASHQHRSSPPASNGMLPPASKPGRGKCTTAATLLDLIKDVEIAISCRKGRSGTAAGDVAFPKGKENLASVLKRYKRRLSNKLIGSK

SEQ ID NO: 19 - DNA, 21 bp, Artificial Sequence
  Synthetic polynucleotide
gaggatggttgttctccggtg

SEQ ID NO: 20 - DNA, 21 bp, Artificial Sequence
  Synthetic polynucleotide
acggagcggtgatcaaagtca

SEQ ID NO: 21 - DNA, 22 bp, Artificial Sequence
  Synthetic polynucleotide
cgttcagcatctggagtttcac

SEQ ID NO: 22 - DNA, 21 bp, Artificial Sequence
  Synthetic polynucleotide
ccatcatcacccttatcttcg